SEQUENCE LISTING (5 sequences)

SEQ ID NO: 1 - protein, 1457 aa, Artificial Sequence
  Description of Artificial Sequence Factor       VIII-BDD
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 1451 aa, Canis familiaris
MQVELYTCCFLCLLPFSLSATRKYYLGAVELSWDYMQSDLLSALHADTSFSSRVPGSLPLTTSVTYRKTVFVEFTDDLFNIAKPRPPWMGLLGPTIQAEVYDTVVIVLKNMASHPVSLHAVGVSYWKASEGAEYEDQTSQKEKEDDNVIPGESHTYVWQVLKENGPMASDPPCLTYSYFSHVDLVKDLNSGLIGALLVCKEGSLAKERTQTLQEFVLLFAVFDEGKSWHSETNASLTQAEAQHELHTINGYVNRSLPGLTVCHKRSVYWHVIGMGTTPEVHSIFLEGHTFLVGNHRQASLEISPITFLTAQTFLMDLGQFLLFCHIPSHQHDGMEAYVKVDSCPEEPQLRMKNNEDKDYDDGLYGSDMDVVSFDDDSSSPFIQIRSVAKKHPKTWVHYIAAEEEDWDYAPSGPTPNDRSHKNLYLNNGPQRIGKKYKKVRFVAYTDETFKTREAIQYESGILGPLLYGEVGDTLLIIFKKQASRPYNIYPHGINYVTPLHTGRLPKGVKHLKDMPILPGEIFKYKWTVTVEDGPTKSDPRCLTRYYSSFINLERDLASGLIGPLLICYKESVDQRGNQMMSDKRNVILFSVLDENRSWYLTEDMQRFLPNADVVQPHDPEFQLSNIMHSINGYVFDNLQLSVCLHEVAYWYILSVGAQTDFLSVFFSGYTFKHKMVYEDTLTLFPFSGETVFMSMENPGLWVLGCHNSDFRNRGMTALLKVSSCNRNIDDYYEDTYEDIPTPLLNENNVIKPRSFSQNPPVSKHHQREITVTTLQPEEDKFEYDDTFSIEMKREDFDIYGDYEDQGLRSFQKKTRHYFIAAVERLWDYGMSRSPHILRNRAQSGDVQQFKKVVFQEFTDGSFTQPLYRGELNEHLGLLGPYIRAEVEDNIVVTFKNQASRPYSFYSSLISYDEDEGQGAEPRRKFVNPNETKIYFWKVQHHMAPTKDEFDCKAWAYFSDVDLEKDVHSGLIGPLLICRSNTLNPAHGRQVTVQEFALVFTIFDETKSWYFTENLERNCRAPCNVQKEDPTLKENFRFHAINGYVKDTLPGLVMAQDQKVRWYLLSMGSNENIHSIHFSGHVFTVRKKEEYKMAVYNLYPGVFETVEMLPSQVGIWRIECLIGEHLQAGMSTLFLVYSKKCQTPLGMASGHIRDFQITASGQYGQWAPKLARLHYSGSINAWSTKDPFSWIKVDLLAPMIIHGIMTQGARQKFSSLYVSQFIIMYSLDGNKWHSYRGNSTGTLMVFFGNVDSSGIKHNIFNPPIIAQYIRLHPTHYSIRSTLRMELLGCDFNSCSMPLGMESKAISDAQITASSYLSSMLATWSPSQARLHLQGRTNAWRPQANNPKEWLQVDFRKTMKVTGITTQGVKSLLISMYVKEFLISSSQDGHNWTLFLQNDKVKVFQGNRDSSTPVRNALEPPLVARYVRLHPQSWAHHIALRLEVLGCDTQQPA

SEQ ID NO: 3 - protein, 1457 aa, Porcine
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYISAEEEDWDYAPAVPSPSDRSYKSLYLNSGPQRIGRKYKKARFVAYTDVTFKTRKAIPYESGILGPLLYGEVGDTLLIIFKNKASRPYNIYPHGITDVSALHPGRLLKGWKHLKDMPILPGETFKYKWTVTVEDGPTKSDPRCLTRYYSSSINLEKDLASGLIGPLLICYKESVDQRGNQMMSDKRNVILFSVFDENQSWYLAENIQRFLPNPDGLQPQDPEFQASNIMHSINGYVFDSLQLSVCLHEVAYWYILSVGAQTDFLSVFFSGYTFKHKMVYEDTLTLFPFSGETVFMSMENPGLWVLGCHNSDLRNRGMTALLKVYSCDRDIGDYYDNTYEDIPGFLLSGKNVIEPRSFAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKECQAPLGMASGRIRDFQITASGQYGQWAPKLARLHYSGSINAWSTKDPHSWIKVDLLAPMIIHGIMTQGARQKFSSLYISQFIIMYSLDGRNWQSYRGNSTGTLMVFFGNVDASGIKHNIFNPPIVARYIRLHPTHYSIRSTLRMELMGCDLNSCSMPLGMQNKAISDSQITASSHLSNIFATWSPSQARLHLQGRTNAWRPRVSSAEEWLQVDLQKTVKVTGITTQGVKSLLSSMYVKEFLVSSSQDGRRWTLFLQDGHTKVFQGNQDSSTPVVNALDPPLFTRYLRIHPTSWAQHIALRLEVLGCEAQDLY

SEQ ID NO: 4 - protein, 24 aa, Artificial Sequence
  Description of Artificial Sequence hF       VIII-BDD Peptide
PRSFSQNPPVLKRHQREITRTTLQ

SEQ ID NO: 5 - protein, 20 aa, Artificial Sequence
  Description of Artificial Sequence hF       VIII-BDD Peptide
PRSFSQNPPVLKEITRTTLQ

(19) United States
(12) Patent Application Publication
(10) Pub. No.: US 2019/0144524 A1
(43) Pub. Date: May 16, 2019

(54) FACTOR VIII VARIANTS, NUCLEIC ACID SEQUENCES, AND METHODS AND USES FOR TREATMENT OF HEMOSTASIS DISORDERS

(71) Applicants: Denise SABATINO, Havertown, PA (US); Giang NGUYEN, Philadelphia, PA (US); The Children's Hospital of Philadelphia, Philadelphia, PA (US)

(72) Inventors: Denise E. SABATINO, Havertown, PA (US); Giang NGUYEN, Philadelphia, PA (US)

(73) Assignee: The Children's Hospital of Philadelphia, Philadelphia, PA (US)

(21) Appl. No.: 16/069,778

(22) PCT Filed: Jan. 13, 2017
(86) PCT No.: PCT/US2017/013461

§ 371 Date: Jul. 12, 2018

Related U.S. Application Data
(60) Provisional application No. 62/278,767, filed on Jan. 14, 2016.
(60) Provisional application No. 62/297,352, filed on Feb. 19, 2016.

(51) Int. Cl. C07K 14/755; A61K 38/37; A61P 7/04; A61K 48/00

(52) U.S. Cl. CPC C07K 14/755; A61K 38/37; A61K 38/00; A61K 48/005; A61P 7/04

(57) ABSTRACT

Factor VIII variants and methods of use thereof are disclosed. In particular embodiments, Factor VIII variants exhibit one or more improvements compared to wild-type Factor VIII proteins, including wild-type Factor VIII proteins with a B-domain deletion (FVIII-BDD). Examples may include enhanced activity or function, secretion at increased levels by cells or are packaged more efficiently into viral vectors.

### RELATED APPLICATIONS

This patent application is the National Phase of International Application No. PCT/US2017/013461, filed Jan. 13, 2017, which designated the U.S. and that International Application was published under PCT Article 21(2) in English, which claims the benefit of priority to U.S. Provisional Patent Application No. 62/297,352, filed Feb. 19, 2016 and U.S. Provisional Patent Application No. 62/278,767, filed Jan. 14, 2016. The entire contents of the foregoing applications are incorporated herein by reference, including all text, tables, sequence listings and drawings.

### SEQUENCE LISTING

The instant application contains a Sequence Listing which has been submitted electronically in ASCII format and is hereby incorporated by reference in its entirety. Said ASCII copy, created on Jul. 11, 2018, is named “CHOP0460589_ST25.txt” and is 38.2 KB in size.

### FIELD OF THE INVENTION

This invention relates to the fields of recombinant coagulation factor production and the treatment of medical disorders associated with aberrant hemostasis. More particularly, the invention provides Factor VIII variants of the invention, the improved variant also exhibiting enhanced function and/or activity over wild-type Factor VIII proteins.

### INTRODUCTION

Several publications and patent documents are cited throughout the specification in order to describe the state of the art to which this invention pertains. Each of these citations is incorporated herein by reference as though set forth in full.

Hemophilia is an X-linked bleeding disorder present in 1 in 5,000 males worldwide. Therapies aimed at increasing clotting factor levels just above 1% of normal are associated with substantial improvement of the severe disease phenotype. Recent clinical trials for AAV-mediated gene transfer for hemophilia B (HB) have demonstrated sustained long-term expression of therapeutic levels of factor IX (FIX) but established that the AAV vector dose may be limiting due to anti-AAV immune responses to the AAV capsid. While these data relate to hemophilia B, 80% of all hemophilia is due to FVIII deficiency, hemophilia A (HA).

Current treatment for this disease is protein replacement therapy that requires frequent infusion of the factor VIII protein. There is an immediate need to achieve sustained therapeutic levels of factor VIII expression so that patients no longer require such frequent protein treatments. Indeed, continuous factor VIII expression would prevent bleeding episodes and may ensure that immune tolerance to the protein is established.

In summary, gene therapy for HA presents 3 distinct challenges: (1) intrinsic properties of human FVIII (hFVIII) make it difficult to express compared to other proteins of similar size (2) the large size of the FVIII cDNA and sequence specific effects are correlated with rearrangements which hamper AAV production and (3) high rates of anti-FVIII antibody (inhibitors) formation in response to protein therapy that occurs in 25-30% of severe (<1% FVIII) HA patients.

The invention provides improved Factor VIII variants useful for treatment in patients in need thereof, such as a patient with HA.

### SUMMARY

Hemophilia A (HA) is an X-linked bleeding disease characterized by deficiency in factor VIII (FVIII), a key component of the coagulation cascade. The FVIII gene contains 26 exons that span 186 kb and is synthesized as a large precursor molecule (2332 amino acids).

Affected individuals commonly suffer joint, muscle, as well as intracranial and intraperitoneal hemorrhages that can be lethal. The normal plasma FVIII level is 100-200 ng/ml, but small amounts of circulating FVIII (˜1-2 ng/ml) are sufficient to have a substantial effect on the clinical course of patients with severe disease. The current treatment for HA patients is protein replacement therapy using recombinant or plasma-derived FVIII. However, these products are only available to ˜20% of the HA population worldwide. The major complication of this therapy is the development of neutralizing antibodies (inhibitors) to FVIII that occurs in 25-30% of patients with severe HA. Since inhibitors render the FVIII protein therapy ineffective, bypass agents (FVIIa) are used to achieve hemostasis, however, these products are very expensive alternatives.

Disclosed herein are factor VIII (FVIII) variants useful in the setting of gene and protein expression systems. Each factor VIII (FVIII) variant can be encoded by a gene, which can optionally include one or more of an expression control (e.g., promoter) element, factor VIII gene and other regulatory features required for expression of the gene, such as introns, ITRs, stop codons, poly A signals, etc.

In accordance with the invention, VIII (FVIII) variants and nucleic acids encoding FVIII variants distinct from wild-type FVIII are provided. Such FVIII variants optionally have a B domain deletion, such as FVIII protein (e.g., human FVIII protein) that lacks most of the B domain (see, e.g., FIG. 1). Such FVIII variants may exhibit increased expression and/or activity (see, e.g., FIGS. 3-6). In addition, FVIII variants may exhibit increased activity and/or stability (see. e.g., FIGS. 7-8). Invention FVIII variants, with or that lacks all or a part of the B-domain, may optionally have a mutated or deleted PACE-furin cleavage recognition site.

In one embodiment, a Factor VIII (FVIII) variant has 1 or 2 amino acids at positions 1657 or 1658 of FVIII protein substituted, modified or deleted compared to wild type FVIII. In another embodiment, a Factor VIII (FVIII) variant has a B domain deletion and has 1 or 2 amino acids at positions 1657 or 1658 of FVIII protein substituted, modified or deleted. In a further embodiment, a Factor VIII (FVIII) variant, optionally with a B-domain or having a B-domain deletion (FVIII-BDD), which, has one or more amino acids at positions 1645 through 1662 of FVIII protein substituted, modified or deleted compared to wild type FVIII with a B-domain or having a B-domain deletion.

In still further embodiments, a Factor VIII (FVIII) variant has 1 to 6 amino acids at positions 1653 to 1658 of FVIII protein substituted, modified or deleted compared to wild type FVIII. In additional embodiments, a FVIII variant comprises a B domain deletion and has 1 to 6 amino acids at positions 1653 to 1658 of FVIII protein substituted, modified or deleted, compared to wild type FVIII comprising a B domain deletion.

In yet further embodiments, a FVIII variant has 1 to 6 amino acids at positions 1657 through 1662 of FVIII protein substituted, modified or deleted compared to wild type FVIII. In yet additional embodiments, a FVIII variant comprises a B domain deletion and has 1 to 6 amino acids at positions 1657 through 1662 of FVIII protein substituted, modified or deleted, compared to wild type FVIII comprising a B domain deletion.

In other embodiments, a FVIII variant has 1 to 10 amino acids at positions 1653 to 1662 of FVIII protein substituted, modified or deleted compared to wild type FVIII. In further other embodiments, a FVIII variant comprises a B domain deletion with one or more amino acids at positions 1657 through 1662 of FVIII protein substituted, modified or deleted compared to wild type FVIII comprising a B domain deletion.

In still other embodiments, a Factor VIII (FVIII) variant has 1 or 2 amino acids at positions 1649 or 1650 of canine FVIII protein substituted, modified or deleted compared to wild type FVIII, a Factor VIII (FVIII) variant has a B domain deletion and has 1 or 2 amino acids at positions 1649 or 1650 of canine FVIII protein substituted, modified or deleted, a Factor VIII (FVIII) variant, optionally with a B-domain or having a B-domain deletion (FVIII-BDD), which, has one or more amino acids at positions 1637-1655 of canine FVIII protein substituted, modified or deleted compared to wild type FVIII with a B-domain or having a B-domain deletion.

In still additional embodiments, a Factor VIII (FVIII) variant has 1 or 2 amino acids at positions 1439 or 1440 of porcine FVIII protein substituted, modified or deleted compared to wild type FVIII, a Factor VIII (FVIII) variant has a B domain deletion and has 1 or 2 amino acids at positions 1439 or 1440 of porcine FVIII protein substituted, modified or deleted, a Factor VIII (FVIII) variant, optionally with a B-domain or having a B-domain deletion (FVIII-BDD), which, has one or more amino acids at positions 1427-1445 of porcine FVIII protein substituted, modified or deleted compared to wild type FVIII with a B-domain or having a B-domain deletion.

In further embodiments, FVIII variants further include a mutated PACE-furin cleavage recognition site. In particular aspects, FVIII variants as set forth herein, such as, but not limited to, FVIII variant with a B domain deletion, and/or 1 or 2 amino acids at positions 1657 or 1658 of FVIII protein, 1649 or 1650 of canine FVIII protein, or 1439 or 1440 of porcine FVIII protein, substituted (e.g., conservative substitution), modified or deleted, and/or one or more amino acids at positions 1645 through 1674 of FVIII protein, 1637 through 1655 of canine FVIII protein, or 1427 through 1445 of porcine FVIII protein, substituted (e.g., conservative substitution), modified or deleted compared to wild type FVIII, also has 1, 2, 3 or all 4 of the codons encoding the PACE/furin cleavage site of FVIII substituted or deleted. In more particular aspects, 1, 2, 3 or all 4 of the amino acids comprising the PACE/furin cleavage site set forth as HHQR or RHQR from positions 1645-1648, positions 1637-1640, or positions 1427-1430 is/are deleted in FVIII variants and/or the codons encoding the PACE/furin cleavage site set forth as HHQR or RHQR from positions 1645-1648, positions 1637-1640, or positions 1427-1430 is/are deleted in FVIII variants.

In still further embodiments, a Factor VIII (FVIII) variant herein has 1, 2, 1 to 4, 1 to 6 or 1 to 10 amino acids substituted, modified or deleted. In particular aspects, a Factor VIII (FVIII) variant herein with 1, 2, 1 to 4, 1 to 6 or 1 to 10 amino acids substituted, modified or deleted is human, canine or porcine FVIII, such as wild type human, canine or porcine FVIII. Optionally, a Factor VIII (FVIII) variant herein with 1, 2, 1 to 4, 1 to 6 or 1 to 10 amino acids substituted, modified or deleted has a B domain deletion, e.g., human, canine or porcine FVIII with a B domain deletion, such as wild type human, canine or porcine FVIII with a B domain deletion.

In more additional embodiments, a FVIII variant has 1, 2, 3 or all 3 amino acids HHQ or RHQ from positions 1645-1647 of human FVIII substituted, modified or deleted. In still more additional embodiments, a FVIII variant has 1, 2, 3 or all 4 amino acids comprising the PACE/furin cleavage site set forth as HHQR or RHQR from positions 1645-1648 of human FVIII substituted, modified or deleted.

In yet additional embodiments, FVIII variants are secreted by a cell in which it is expressed at least 1-5 fold, or 1.5-3-fold higher than secretion of a wild type FVIII or wild-type FVIII comprising a B domain deletion wherein 3 or all 4 of the amino acids comprising the PACE/furin cleavage site set forth as HHQR or RHQR from positions 1645-1648, positions 1637-1640, or positions 1427-1430 in the wild type FVIII or wild-type FVIII comprising a B domain deletion is/are substituted (e.g., conservative substitution), modified or deleted.

In particular aspects, a FVIII variant has 1 to 6 amino acids at positions 1653 to 1658 of human FVIII protein substituted or deleted, compared to wild type FVIII comprising a B domain deletion. In particular aspects, a FVIII variant has 1 to 6 amino acids at positions 1657 through 1662 of human FVIII protein substituted or deleted, compared to wild type FVIII comprising a B domain deletion. In particular aspects, a FVIII variant has 1 to 6 amino acids at positions 1659 through 1662 of human FVIII protein substituted or deleted, compared to wild type FVIII comprising a B domain deletion.

In particular aspects, a FVIII variant has a threonine deleted at position 1653; and/or wherein said variant has a threonine deleted at position 1654; and/or wherein said variant has a leucine deleted at position 1655; and/or, wherein said variant has a glutamine deleted at position 1656. In particular aspects, a FVIII variant has a glutamic acid substituted for a glutamine at position 1659; and/or wherein said variant has an aspartic acid substituted for a glutamic acid at position 1660; and/or wherein said variant has a lysine substituted for a glutamic acid at position 1661; and/or, wherein said variant has a phenylalanine substituted for a leucine at position 1662. In particular aspects, a FVIII variant has a glutamic acid deleted at position 1659; and/or wherein said variant has an aspartic acid deleted at position 1660; and/or wherein said variant has a lysine deleted at position 1661; and/or, wherein said variant has a phenylalanine deleted at position 1662.

In further particular aspects, a FVIII variant has a proline at position 1657; a FVIII variant has a proline substituted for a serine at position 1657, position 1649; or position 1439; a FVIII variant has a glutamic acid at position 1658, position 1650; or position 1440; a FVIII variant has a glutamic acid substituted for an aspartic acid at position 1658, position 1650; or position 1440; a FVIII variant has a proline at position 1657, position 1649; or position 1439 and an aspartic acid at position 1658, position 1650; or position 1440; and/or a FVIII variant has a proline substituted for a serine at position 1657, position 1649; or position 1439 and a glutamic acid substituted for an aspartic acid at position 1658, position 1650; or position 1440.

In still other embodiments, a FVIII variant has one or more amino acid substitutions and/or deletions as shown in Table 3. In particular aspects, a FVIII variant has one or more or the same amino acid substitutions and/or deletions shown in Table 3 (denoted as hFVIII-S1657P/D1658E (SP/DE); hFVIII-Δ3-S1657P (Δ3-SP); hFVIII-Δ3-D1658E (Δ3-DE); hFVIII-Δ3-S1657P/D1658E (Δ3-SP/DE); hFVIII-Δ3-del1657-58 (Δ3-del57-58); hFVIII-Δ3-del1653-58 (43-del53-58); or hFVIII-Δ3-del1657-62 (Δ3-del57-62); hFVIII-Δ3-del1657PEEDKF1662 (Δ3-457-62)).

In still additional embodiments, a FVIII variant exhibits greater biological activity when compared to wild type FVIII or when compared to wild type FVIII comprising a B domain deletion (e.g., as determined by a clotting assay or reduced bleeding in a FVIII assay or FVIII deficiency model). In another embodiment, a FVIII variant exhibits greater expression when compared to wild type FVIII or when compared to expression of wild type FVIII comprising a B domain deletion.

In additional embodiments, FVIII variants are encoded by nucleic acid sequences, such as optimized nucleic acid sequences. In particular aspects, optimized nucleic acid sequences have one or more leucine codons changed to CTG compared to TTA, TTG, CTT, CTC or CTA in wild type FVIII encoding nucleic acid. In further aspects, nucleic acids encoding FVIII variants have 2-5, 5-10, 10-20, 20-50, 50-100, 100-250, 250-500, 500-750 or 750-850 CTG leucine codons modified from TTA, TTG, CTT, CTC or CTA leucine codons in wild type FVIII encoding nucleic acid. In yet additional aspects, nucleic acids encoding FVIII variants have greater than 85% CTG leucine codons modified from TTA, TTG, CTT, CTC or CTA leucine codons in wild type FVIII encoding nucleic acid. In still further aspects, nucleic acids encoding FVIII variants have all CTG leucine codons modified from TTA, TTG, CTT, CTC or CTA leucine codons in wild type FVIII encoding nucleic acid. In particular aspects, nucleic acids encoding FVIII variants have between about 50-59%, or 50-56%, or 50-53% GC content. In other aspects, nucleic acids encoding FVIII variants have one or more AAG lysine codons compared to AAA lysine codons in wild type FVIII encoding nucleic acid.

In still additional embodiments, FVIII variants are at least 75% identical to wild type human FVIII nucleic acid or wild type human FVIII nucleic acid comprising a B domain deletion. In more particular aspects, FVIII variants are at least about 75% identical (e.g., about 75%, 76%, 77%, 78%, 79%, 80%, 81%, 82%, 83%, 84%, 85%, 86%, 87%, 88%, 89%, 90%, etc. up to 100% identical) to wild type human FVIII nucleic acid or wild type human FVIII nucleic acid comprising a B domain deletion.

In various embodiments, FVIII variants are mammalian, such as human, canine or porcine. Such mammalian FVIII variants including human forms may be based upon wild type FVIII or wild type FVIII comprising a B domain deletion.

In accordance with the invention, also provided are expression vectors that include nucleic acid encoding FVIII variants as set forth herein. In particular embodiments, an expression vector comprises a viral vector such as, but not limited to, an adenovirus-associated virus (AAV) vector, a retroviral vector, an adenoviral vector, a plasmid, or a lentiviral vector.

Expression vectors can include additional components or elements. In particular embodiments, an expression vector such as, but not limited to, AAV vector further includes an intron, an expression control element, one or more AAV inverted terminal repeats (ITRs) (e.g., any of: AAV1, AAV2, AAV3, AAV4, AAV5, AAV6, AAV7, AAV8, AAV9, AAV10, AAV11, Rh10, Rh74 or AAV-2i8 AAV serotypes, or a combination thereof) and/or a filler polynucleotide sequence. In particular aspects, an intron is within or flanks a nucleic acid encoding FVIII variant, and/or an expression control element is operably linked to the nucleic acid encoding FVIII variant, and/or an AAV ITR(s) flanks the 5′ or 3′ terminus of the nucleic acid encoding FVIII variant, and/or a filler polynucleotide sequence flanks the 5′ or 3′terminus of the FVIII encoding nucleic acid variant.

In particular aspects, an expression control element comprises a constitutive or regulatable control element, or a tissue-specific expression control element or promoter. In more particular aspects, an expression control element comprises an element that confers expression in liver. In further particular aspects, a promoter comprises a TTR promoter, such as mutant TTR promoter (SEQ ID NO:8).

In accordance with the invention, additionally provided are host cells expressing the FVIII variants as set forth herein. In particular embodiments, a host cell includes a nucleic acid encoding FVIII variant or an expression vector comprising a nucleic acid encoding FVIII variant. In particular aspects, such host cells produce FVIII variant protein encoded by the nucleic acid and FVIII protein produced is recovered. Such FVIII protein produced by the cells, optionally isolated and/or purified, can be administered to a subject as set forth herein.

In accordance with the invention, further provided are virus vectors that include the nucleic acid encoding FVIII variants or the expression vectors comprising the nucleic acid encoding FVIII variants. In particular embodiments, a viral vector comprises an AAV vector. In particular aspects, an AAV vector comprises a VP1, VP2 and/or VP3 capsid sequence having 75% or more sequence identity (e.g., 80%, 85%, 90%, 95%, 96%, 97%, 98%, 99%, 99.1%, 99.2%, 99.3%, 99.4%, 99.5%, 99.6%, 99.7%, 99.8%, 99.9%, 99.95%, etc.) to AAV1, AAV2, AAV3, AAV4, AAV5, AAV6, AAV7, AAV8, AAV9, AAV10, AAV11, Rh10, Rh74 or AAV-2i8 VP1, VP2 and/or VP3 sequences. In more particular aspects, an AAV vector comprises a VP1, VP2 and/or VP3 capsid sequence selected from any of: AAV1, AAV2, AAV3, AAV4, AAV5, AAV6, AAV7, AAV8, AAV9, AAV10, AAV11, Rh10, Rh74 and AAV-2i8 AAV serotypes.

In accordance with the invention, yet additionally provided are compositions including FVIII variants and nucleic acid encoding FVIII variants as set forth herein. In particular embodiments, pharmaceutical compositions include a FVIII variant protein, or a FVIII variant bearing expression vector, or a virus or AAV vector, in a biologically compatible carrier or excipient. Such pharmaceutical compositions optionally include empty capsid AAV (e.g., lack vector genome comprising FVIII encoding nucleic acid variant). In additional particular embodiments, FVIII variants, nucleic acid encoding FVIII variants, expression vectors, or virus or AAV vectors are encapsulated in a liposome or mixed with phospholipids or micelles.

In accordance with the invention, still further provided are methods for delivering or transferring a FVIII variant into a mammal or a mammalian cell. In one embodiment, a method includes administering FVIII variant, or contacting nucleic acid encoding a FVIII variant, an expression vector comprising nucleic acid encoding FVIII variant, or a virus or AAV vector comprising a nucleic acid encoding FVIII variant to a mammal or mammalian cell, thereby delivering or transferring the FVIII variant or nucleic acid sequence into the mammal or mammalian cell. Such methods introduce nucleic acid encoding FVIII variants into a mammalian cell in culture or in a subject (e.g., a patient).

Methods of the invention also include treating mammalian subjects (e.g., patients) such as humans in need of Factor VIII (the human produces an insufficient amount of Factor VIII protein, or a defective or aberrant Factor VIII protein). In one embodiment, a method of treating a mammal in need of Factor VIII, includes: providing a FVIII variant or nucleic acid encoding FVIII variant, or an expression vector comprising nucleic acid encoding FVIII variant, or a virus or AAV vector comprising a nucleic acid encoding FVIII variant; and administering an amount of the FVIII variant or nucleic acid encoding FVIII variant, or an expression vector comprising nucleic acid encoding FVIII variant, or a virus or AAV vector comprising a nucleic acid encoding FVIII variant to the mammalian subject such that Factor VIII variant or Factor VIII variant encoded by the nucleic acid, is expressed in the mammalian subject.

In another embodiment, a method for treatment of a hemostasis related disorder in a patient in need thereof (e.g., the patient produces an insufficient amount of Factor VIII protein, or a defective or aberrant Factor VIII protein) includes administration of a therapeutically effective amount of a FVIII variant or nucleic acid encoding FVIII variant, or an expression vector comprising nucleic acid encoding FVIII variant, or a virus or AAV vector comprising a nucleic acid encoding FVIII variant in a biologically acceptable carrier to the patient.

In particular aspects of the invention methods, Factor VIII is provided or expressed at levels having a beneficial or therapeutic effect on the mammal; and/or Factor VIII is expressed in a cell, tissue or organ of the mammal. Such aspects include delivery or introduction of FVIII encoding nucleic acid variant into a tissue or organ such as liver. Such aspects also include introduction of nucleic acid encoding FVIII variant into a secretory cell. Such aspects further include introduction of nucleic acid encoding FVIII variant into an endocrine cell or an endothelial cell. Such aspects additionally include introduction of encoding nucleic acid FVIII variant into a hepatocyte, a sinusoidal endothelial cell, a megakaryocyte, a platelet or hematopoetic stem cell.

Candidate subjects (e.g., a patient) and mammals (e.g., humans) for administration (e.g., delivery) of a FVIII variant or nucleic acid encoding FVIII variant, or an expression vector comprising nucleic acid encoding FVIII variant, or a virus or AAV vector comprising a nucleic acid encoding FVIII variant include those having or those at risk of having a disorder such as: hemophilia A, von Willebrand diseases and bleeding associated with trauma, injury, thrombosis, thrombocytopenia, stroke, coagulopathy, disseminated intravascular coagulation (DIC) or over-anticoagulation treatment disorder.

Candidate subjects (e.g., a patient) and mammals (e.g., humans) for administration (e.g., delivery) of a FVIII variant or a nucleic acid encoding FVIII variant, or an expression vector comprising a nucleic acid encoding FVIII variant, or a virus or AAV vector comprising a nucleic acid encoding FVIII variant include those sero-negative for AAV antibodies, as well as those having or those at risk of developing AAV antibodies. Such subjects (e.g., a patient) and mammals (e.g., humans) may be sero-negative or sero-positive for an AAV1, AAV2, AAV3, AAV4, AAV5, AAV6, AAV7, AAV8, AAV9, AAV10, AAV11, AAV-Rh10 or AAV-Rh74 serotype.

Methods of the invention therefore further include administering empty capsid AAV to said mammal or said patient. In particular embodiments, empty capsid of AAV1, AAV2, AAV3, AAV4, AAV5, AAV6, AAV7, AAV8, AAV9, AAV10, AAV11, AAV-12, AAV-Rh10 and/or AAV-Rh74 serotype is further administered to the mammal or patient.

Methods of administration (e.g., delivery) in accordance with the invention include any mode of contact or delivery, ex vivo or in vivo. In particular embodiments administration (e.g., delivery) is: intravenously, intraarterially, intramuscularly, subcutaneously, intra-cavity, intubation, or via catheter.

In accordance with the invention, still also provided are methods for producing FVIII variants. In one embodiment, a method includes expressing in a cell a FVIII variant as set forth herein, and recovering said FVIII variant protein produced by the cells. In particular aspects, FVIII variant protein produced by the cells is purified and/or isolated.

### DESCRIPTION OF DRAWINGS

FIG. 1 shows Human FVIII expression constructs. This expression cassette contains a short version of the hepatic control region (HCR) of the human apolprotein ExxC-1 gene locus, the human alpha-1 antitrypsin promoter (hAAT), the hFVIII-BDD form with the B-domain junction at S743 and Q1630 and an SV40 polyadenylation signal. The human factor VIII cDNA is the wild type sequence and is not codon-optimized. These FVIII constructs are identical except for the variant introduced at the PACE-furin cleavage recognition site (1645-1648) or the variant that includes both a PACE-furin variant and an a3 variant.

FIG. 2 shows Human factor VIII expression cassettes. The expression cassettes contain a short version of the hepatic control region (HCR) of the human apolprotein ExxC-1 gene locus, the human alpha-1 antitrypsin promoter (hAAT), the hFVIII-BDD form with the B-domain junction at S743 and Q1630 and an SV40 polyadenylation signal. The human factor VIII cDNA is the wild type sequence and is not codon-optimized. The PACE-furin variant that deletes residues 1645-1647 (43) and modifications in the a3 region at positions 1657-1658 were introduced into the human FVIII. The S1657P (SP) replaces the serine residue (S) with a proline residue at this position (P). The D1658E (DE) replaces the aspartic acid residue (D) with the glutamic acid residue (E). The SP/DE variant replaces both of residues (S and D) with residues (P and E).

FIG. 3 shows Human FVIII antigen levels in the circulation after AAV administration of hFVIII variants. AAV8 was delivered to hemA/CD4KO mice (5×10e11vg/mouse)(n=4/group) as described in Example 1. hFVIII antigen levels were determined at week 2 through week 12 by a human FVIII specific ELISA.

FIG. 4 shows hFVIII antigen levels 8 weeks post-AAV administration AAV8 was delivered to hemA/CD4KO mice (5×10e11vg/mouse)(n=4/group). hFVIII antigen levels were determined by a human FVIII specific ELISA. Statistical analysis using one-way ANOVA, Tukey post-test, ** p<0.01, * p<0.05.

FIG. 5 shows Human FVIII antigen levels in the circulation after AAV administration of hFVIII variants. In a second study (Study 2), AAV8 was delivered to hemA/CD4KO mice (5×10e11vg/mouse) (n=5/group). hFVIII antigen levels were determined at weeks 2 through week 12 by a human FVIII specific ELISA.

FIG. 6 shows hFVIII antigen levels 2 weeks post-AAV administration AAV8 was delivered to hemA/CD4KO mice (5×10e11vg/mouse)(n=4/group). hFVIII antigen levels were determined by a human FVIII specific ELISA.

FIG. 7 shows hFVIII antigen levels 8 weeks post-AAV administration AAV8 was delivered to hemA/CD4KO mice (5×10e11vg/mouse)(n=5/group). hFVIII antigen levels were determined by a human FVIII specific ELISA. Statistical analysis using one-way ANOVA, Tukey post-test *** p<0.001.

FIG. 8 shows in vivo hemostatic challenge using the tail clip assay. The AAV treated hemA/CD4KO mice (Study 2) were challenged by tail clip assay at 6 weeks post-vector administration. The tails were warmed in saline (37° C.) for 10 minutes followed by complete tail transection. The blood was collected into the tube of warm saline for 10 minutes. The blood sample was centrifuged for 15 minutes at 525 g and the remaining red blood cell pellet was lysed with 6 ml of lysis buffer (NH4Cl 0.15M, KHCO3 10 nM, EDTA 1 mM) for 10 minutes. The sample was centrifuged to eliminate cellular debris. The absorbance at 575 nm of the supernatant containing hemoglobin is determined, and converted to total blood loss (μl) based on a standard curve of whole blood loss (Ivanciu L Nat Biotechnol. 2011). hFVIII-BDD (SQ), Wild type mice (WT mice), hemophilia A mice (HA mice).

FIG. 9 shows hFVIII sequence from amino acids 1642 through 1689.

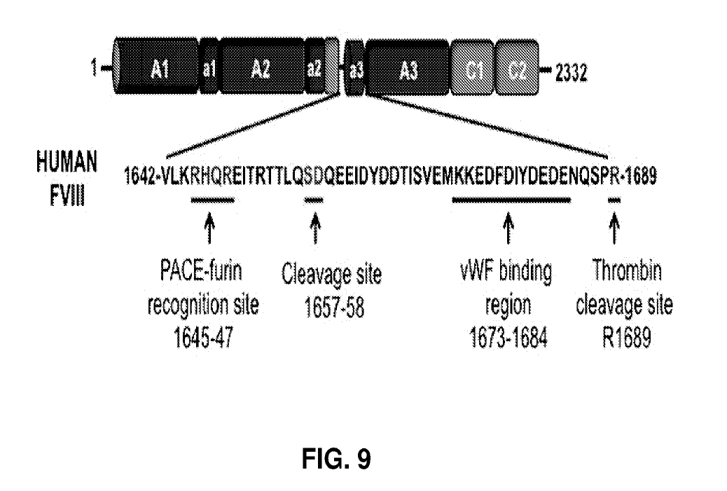

FIG. 10 shows SDS-PAGE gel analysis of a3 variants. Purified protein (3 μg) was loaded on the gel under reducing conditions. The percent of single chain (SC) was determined by optical densitometry. Variants Δ3, Δ3 SP/DE are shown alongside hFVIII-BDD and cFVIII.

FIG. 11 shows SDS-PAGE gel analysis of a3 variants. Purified protein (3 μg) was loaded on the gel under reducing conditions. The percent of single chain (SC) was determined by optical densitometry. Variants Δ3, SP/DE and 43 SP/DE are shown alongside hFVIII-BDD and cFVIII.

FIG. 12 shows comparison of the activity of hFVIII variants. (A) One-stage aPTT. (B) Two-stage aPTT. In the two-stage assay the proteins were pre-activated with thrombin prior to aPTT.

FIG. 13 shows optimized human FVIII expression constructs. This expression cassette contains a modified version of the transthyretin (TTRm) promoter (222 bp), a synthetic intron (108 bp), the hFVIII-BDD form with the B-domain junction at S743 and Q1630 (4374 bp) and a polyadenylation signal (46 bp). The human factor VIII cDNA is the wild type sequence and is not codon-optimized. These FVIII constructs are identical except for the variant introduced at the PACE-furin cleavage recognition site (1645-1648) or the variant that includes both a PACE-furin variant and an a3 variant.

FIG. 14 shows optimized human factor VIII expression cassettes. These expression cassettes use a modified transthyretin (TTRm) promoter and the wild type hFVIII cDNA sequence. The PACE-furin variant that deletes residues 1645-1647 (43) and a modification in the a3 region at position 1657-1658 were introduced into the human FVIII. The SP/DE is an S1657P and D1658E modification. This variant replaces the human residue (serine, S) with the canine residue (proline, P) at position 1657 and also replaces the human residue (aspartic acid, D) with the canine residue (glutamic acid, E) at position 1658. The Δ3-del53-58 variant utilizes the 43 furin variant with a deletion of residues 1653-165 of the a3 region. The Δ3-del57-62 variant uses the Δ3 furin variant with a deletion of residues 1657-1662. The Δ3-457-62 variant uses the Δ3 furin variant but replaces the human amino acid sequence at position 1657-1662 with the canine sequence at this position.

FIG. 15 shows human FVIII antigen levels in the circulation after AAV administration of hFVIII variants. AAV8-TTRm-hFVIII optimized expression cassette described in FIG. 14 was used to deliver the hFVIII-BDD and the hFVIII variants to hemophilia A/CD4KO mice (1×10e11vg/mouse)(n=4 mice/group). hFVIII antigen levels were determined by a human FVIII specific ELISA.

FIG. 16 shows hFVIII antigen levels 4 weeks post-AAV administration. AAV8-TTRm-hFVIII was delivered to hemophilia A/CD4KO mice (1×10e11vg/mouse)(n=4 mice/group). hFVIII antigen levels were determined by a human FVIII specific ELISA. * significantly different than BDD (One-way ANOVA).

FIG. 17 shows in vivo hemostatic challenge using tail clip bleeding assay. Tail clip assay was performed at 6 weeks after AAV8-TTRm-hFVIII delivery described in FIG. 15 in hemophilia A/CD4KO mice (1×10e11vg/mouse)(n=4 mice/group). The AAV treated hemophilia A/CD4KO mice were compared to hemophilia A mice and wild type mice. The tail vein was transected and blood was collected from the tail for ten minutes in warm saline. Blood loss was quantitated by lysing the red blood cell pellet and measuring absorbance at 575 nm.

### DETAILED DESCRIPTION

Disclosed herein are Factor VIII (FVIII) variants, such as human Factor VIII (hFVIII) variants distinct from wild-type hFVIII. Such FVIII variants exhibit increased function or activity, and/or are expressed at increased levels in cells and/or animals, which in turn can provide increased FVIII function or activity, or FVIII protein levels, in vivo. Also disclosed herein are nucleic acids that encode FVIII variants. Further disclosed herein are human FVIII variants having higher stability and/or biological activity in vitro and/or in vivo, and nucleic acids encoding such human FVIII variants. Such FVIII variants include those with the B-domain or having a B-domain deletion (FVIII-BDD). Such variants optionally exhibit 1) increased expression in cells and/or animals; 2) increased function or activity, as reflected by increased clotting, for example; 3) increased stability; and/or 4) achieve therapeutic effect at lower doses than native hFVIII.

As used herein, the terms “variant” or “modify” and grammatical variations thereof, mean that a polypeptide, nucleic acid, or subsequence thereof deviates from a reference sequence. Modified and variant sequences may therefore have substantially the same, greater or less expression, activity or function than a reference sequence, but at least retain partial activity or function of the reference sequence. A particular example of a variant is a FVIII protein having one or more amino acid substitutions, deletions, insertions and/or additions. A further particular example of a variant is a FVIII-BDD protein having one or more additional amino acid substitutions, deletions, insertions and/or additions.

The “polypeptides,” “proteins” and “peptides” encoded by the “nucleic acid” or “polynucleotide” sequences,” include full-length native (FVIII) sequences, as with naturally occurring wild-type proteins, as well as functional subsequences, modified forms or sequence variants so long as the subsequence, modified form or variant retain some degree of functionality of the native full-length protein. For example, a FVIII protein can have a B-domain deletion (FVIII-BDD), which is all or a part of the B-domain deleted, as set forth herein, and retain clotting function. In a particular embodiment, a FVIII protein has most of the B-domain deleted and leaves a residual sequence. In a particular aspect, FVIII-BDD has 14 or fewer amino acid residues of the B-domain left. A further deletion of 4 residues that is the putative PACE-furin site from the 14 residues leaves a PACE-furin deletion which has only 10 amino acid residues of the B-domain left. In more particular aspects, an exemplary hFVIII-BDD has 14 amino acid residues left of the B-domain (underlined):

PRSFSQNPPVLKRHQREITRTTLQ.

Another exemplary hFVIII-BDD-that has 10 amino acid residues left of the B-domain (underlined), but lacks a PACE furin site, has the sequence:

PRSFSQNPPVLKEITRTTLQ.

Accordingly, such polypeptides, proteins and peptides, and corresponding encoding nucleic acid sequences, can be but are not required to be identical to the endogenous protein that is defective, or whose expression is insufficient, or deficient in the treated mammal.

The terms “polynucleotide” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides include naturally occurring, synthetic, and intentionally modified or altered polynucleotides (e.g., variant nucleic acid). Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

A “nucleic acid” or “polynucleotide” variant refers to a modified sequence which has been genetically altered compared to wild-type. The sequence may be genetically modified to encode a variant protein. Alternatively, the sequence may be genetically modified without altering the encoded protein sequence. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of the protein (FVIII) encoded thereby, and some codons of the nucleic acid variant will be changed which in turn changes the amino acids of the protein (FVIII) encoded thereby.

Accordingly, a Factor VIII (FVIII) variant can refer to a modified FVIII which has been genetically altered such that the encoded protein is the same or differs from a wild-type FVIII. Such a variant having different amino acids can be referred to as a “Factor VIII (FVIII) protein variant.” Alternatively, a Factor VIII (FVIII) variant can refer to a modified FVIII which has been genetically altered such that the one or more of the amino acids in the encoded protein are the same as a wild-type FVIII, or FVIII-BDD. Such a variant can be referred to as a “Factor VIII (FVIII) encoding nucleic acid variant.”

Accordingly a “Factor VIII (FVIII) variant” can mean a modified FVIII protein such that the modified protein has an amino acid alteration compared to wild-type FVIII or an amino acid alteration compared to wild-type FVIII-BDD, which optionally exhibits an increase in function or activity and/or stability compared to wild-type FVIII or wild-type FVIII-BDD. Examples of such particular FVIII protein modifications are genetic modifications that lead to cleavage recognition site mutations, deletions or substitutions in the FVIII protein.

Particular examples of FVIII modifications are FVIII variants which exhibit increased function or activity, and/or are expressed at increased levels, as compared to wild-type FVIII or as compared to FVIII-BDD. When comparing activity and/or stability, if the FVIII variant protein retains the B-domain, it is appropriate to compare it to wild-type FVIII; and if the FVIII variant protein has a B-domain deletion, it is appropriate to be compared to wild-type FVIII that also has a B-domain deletion. Thus, for such comparisons, for example, when comparing function or activity of a FVIII variant that retains the B-domain, it is appropriate to compare it to wild-type FVIII; and when the FVIII variant has a B-domain deletion, it is appropriate to compare it to wild-type FVIII that also has a B-domain deletion.

Non-limiting examples of modifications include one or more amino acid or nucleotide substitutions, deletions, insertions or additions (e.g., 1, 2, 3, 4, 5, 6, 7, 8, 9 or 10 (e.g., 1, 2, 1-4, 1-6, 1-10), or from 2-4, 4-6, 6-10, 10-15, 15-20, 20-25, 25-30, 30-40, 40-50, 50-100, 100-150, 150-200, 200-250, 250-500, 500-750, 750-850 or more residues or nucleotides). An example of an amino acid modification is a non-conservative or a conservative amino acid substitution or a deletion (e.g., subsequences or fragments, or deletion of cleavage site) of a reference sequence, e.g. FVIII. A specific example of a substitution of an amino acid or nucleotide is where either or both of residues 1657-58 of human FVIII, or 1649-50 of canine FVIII, or 1439-40 of porcine FVIII is/are substituted or deleted. A further specific example of a substitution of an amino acid or nucleotide is where any or all of the encoded residues at positions 1645-48,1637-1640 or 1427-1430 of hFVIII is/are substituted or deleted. In particular embodiments, a modified or variant sequence retains at least part of a function or activity of unmodified sequence.

An example of a nucleic acid modification is codon optimization, e.g., for a leucine codon that is not CTG to be modified to CTG, or a lysine codon that is not AAG to be modified to AAG. Another example of a nucleic acid codon optimization modification is increasing GC content. In particular aspects, a nucleic acid sequence encoding human FVIII variant protein has 1-5% more GC content than native sequence encoding human Factor FVIII (e.g., 1, 2, 3, 4 or 5% more GC content); or has 5-10% more GC content than native (wild-type) sequence encoding human Factor FVIII (e.g., 5, 6, 7, 8, 9 or 10% more GC content); or has 10-15% more GC content than native (wild-type) sequence encoding human Factor FVIII (e.g., 10, 11, 12, 13, 14 or 15% more GC content). In particular aspects, a nucleic acid sequence encoding canine FVIII variant protein has 1-5% more GC content than native sequence encoding canine Factor FVIII (e.g., 1, 2, 3, 4 or 5% more GC content); or has 5-10% more GC content than native (wild-type) sequence encoding canine Factor FVIII (e.g., 5, 6, 7, 8, 9 or 10% more GC content); or has 10-15% more GC content than native (wild-type) sequence encoding canine Factor FVIII (e.g., 10, 11, 12, 13, 14 or 15% more GC content). In particular aspects, a nucleic acid sequence encoding porcine FVIII variant protein has 1-5% more GC content than native sequence encoding porcine Factor FVIII (e.g., 1, 2, 3, 4 or 5% more GC content); or has 5-10% more GC content than native (wild-type) sequence encoding porcine Factor FVIII (e.g., 5, 6, 7, 8, 9 or 10% more GC content); or has 10-15% more GC content than native (wild-type) sequence encoding porcine Factor FVIII (e.g., 10, 11, 12, 13, 14 or 15% more GC content).

All mammalian and non-mammalian forms of proteins and encoding nucleic acids, including other mammalian forms of the FVIII proteins and FVIII nucleic acid disclosed herein are expressly included, either known or unknown. Thus, the invention includes proteins and genes from non-mammals, mammals other than humans, and humans, which genes and proteins function in a substantially similar manner to the FVIII (e.g., human, canine, porcine, etc.) proteins and genes described herein.

The term “vector” refers to small carrier nucleic acid molecule, a plasmid, virus (e.g., AAV vector), or other vehicle that can be manipulated by insertion or incorporation of a nucleic acid. Such vectors can be used for genetic manipulation (i.e., “cloning vectors”), to introduce/transfer polynucleotides into cells, and to transcribe or translate the inserted polynucleotide in cells. An “expression vector” is a specialized vector that contains a gene or nucleic acid sequence with the necessary regulatory regions needed for expression in a host cell. A vector nucleic acid sequence generally contains at least an origin of replication for propagation in a cell and optionally additional elements, such as a heterologous polynucleotide sequence, expression control element (e.g., a promoter, enhancer), intron, ITR(s), selectable marker (e.g., antibiotic resistance), polyadenylation signal.

A viral vector is derived from or based upon one or more nucleic acid elements that comprise a viral genome. Particular viral vectors include lentivirus, pseudo-typed lentivirus and parvo-virus vectors, such as, but not limited to, adeno-associated virus (AAV) vectors. Also provided are vectors comprising a nucleic acid sequence encoding a FVIII variant polypeptide.

The term “recombinant,” as a modifier of vector, such as recombinant viral, e.g., lenti- or parvo-virus (e.g., AAV) vectors, as well as a modifier of sequences such as recombinant polynucleotides and polypeptides, means that the compositions have been manipulated (i.e., engineered) in a fashion that generally does not occur in nature. A particular example of a recombinant vector, such as an AAV vector would be where a polynucleotide that is not normally present in the wild-type viral (e.g., AAV) genome is inserted within the viral genome. An example of a recombinant polynucleotide would be where a nucleic acid (e.g., gene) encoding a FVIII protein is cloned into a vector, with or without 5′, 3′ and/or intron regions that the gene is normally associated within the viral (e.g., AAV) genome. Although the term “recombinant” is not always used herein in reference to vectors, such as viral and AAV vectors, as well as sequences such as polynucleotides, recombinant forms including polynucleotides, are expressly included in spite of any such omission.

A recombinant viral “vector” or “AAV vector” is derived from the wild type genome of a virus, such as AAV by using molecular methods to remove the wild type genome from the virus (e.g., AAV), and replacing with a non-native nucleic acid, such as a FVIII encoding nucleic acid variant sequence. Typically, for AAV one or both inverted terminal repeat (ITR) sequences of AAV genome are retained in the AAV vector. A “recombinant” viral vector (e.g., AAV) is distinguished from a viral (e.g., AAV) genome, since all or a part of the viral genome has been replaced with a non-native sequence with respect to the viral (e.g., AAV) genomic nucleic acid such as FVIII encoding nucleic acid variant sequence. Incorporation of a non-native sequence therefore defines the viral vector (e.g., AAV) as a “recombinant” vector, which in the case of AAV can be referred to as a “rAAV vector.”

A recombinant vector (e.g., lenti-, parvo-, AAV) sequence can be packaged—referred to herein as a “particle” for subsequent infection (transduction) of a cell, ex vivo, in vitro or in vivo. Where a recombinant vector sequence is encapsidated or packaged into an AAV particle, the particle can also be referred to as a “rAAV.” Such particles include proteins that encapsidate or package the vector genome. Particular examples include viral envelope proteins, and in the case of AAV, capsid proteins.

A vector “genome” refers to the portion of the recombinant plasmid sequence that is ultimately packaged or encapsidated to form a viral (e.g., AAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsidated into virus (e.g., AAV) particles. Thus, a vector “genome” refers to the nucleic acid that is packaged or encapsidated by virus (e.g., AAV).

A “transgene” is used herein to conveniently refer to a nucleic acid that is intended or has been introduced into a cell or organism. Transgenes include any nucleic acid, such as a gene that encodes a polypeptide or protein (e.g., Factor VIII variant).

In a cell having a transgene, the transgene has been introduced/transferred by way of vector, such as AAV, “transduction” or “transfection” of the cell. The terms “transduce” and “transfect” refer to introduction of a molecule such as a nucleic acid into a cell or host organism. The transgene may or may not be integrated into genomic nucleic acid of the recipient cell. If an introduced nucleic acid becomes integrated into the nucleic acid (genomic DNA) of the recipient cell or organism it can be stably maintained in that cell or organism and further passed on to or inherited by progeny cells or organisms of the recipient cell or organism. Finally, the introduced nucleic acid may exist in the recipient cell or host organism extrachromosomally, or only transiently.

A “transduced cell” is a cell into which a transgene has been introduced. Accordingly, a “transduced” cell (e.g., in a mammal, such as a cell or tissue or organ cell), means a genetic change in a cell following incorporation of an exogenous molecule, for example, a nucleic acid (e.g., a transgene) into the cell. Thus, a “transduced” cell is a cell into which, or a progeny thereof in which an exogenous nucleic acid has been introduced. The cell(s) can be propagated and the introduced protein expressed, or nucleic acid transcribed. For gene therapy uses and methods, a transduced cell can be in a subject.

An “expression control element” refers to nucleic acid sequence(s) that influence expression of an operably linked nucleic acid. Control elements, including expression control elements as set forth herein such as promoters and enhancers. Vector sequences including AAV vectors can include one or more “expression control elements.” Typically, such elements are included to facilitate proper heterologous polynucleotide transcription and if appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but may also act in trans.

Expression control can be effected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances. Nevertheless, owing to the length limitations of certain vectors, such as AAV vectors, expression control elements will typically be within 1 to 1000 nucleotides from the transcribed nucleic acid.

Functionally, expression of operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed sequence e.g., Factor VIII (FVIII) encoding nucleic acid variant. A promoter typically increases an amount expressed from operably linked nucleic acid as compared to an amount expressed when no promoter exists.

An “enhancer” as used herein can refer to a sequence that is located adjacent to the heterologous polynucleotide. Enhancer elements are typically located upstream of a promoter element but also function and can be located downstream of or within a sequence (e.g., Factor VIII (FVIII) encoding nucleic acid variant). Hence, an enhancer element can be located upstream or downstream, e.g., within 100 base pairs, 200 base pairs, or 300 or more base pairs of Factor VIII (FVIII) encoding nucleic acid variant. Enhancer elements typically increase expressed of an operably linked nucleic acid above expression afforded by a promoter element.

An expression construct may comprise regulatory elements which drive expression in a particular cell or tissue type. Expression control elements (e.g., promoters) include those active in a particular tissue or cell type, referred to herein as a “tissue-specific expression control elements/promoters.” Tissue-specific expression control elements are typically active in specific cell or tissue (e.g., liver). Expression control elements are typically active in particular cells, tissues or organs because they are recognized by transcriptional activator proteins, or other regulators of transcription, that are unique to a specific cell, tissue or organ type. Such regulatory elements are known to those of skill in the art (see, e.g., Sambrook et al. (1989) and Ausubel et al. (1992)).

The incorporation of tissue specific regulatory elements in the expression constructs of the invention provides for at least partial tissue tropism for the expression of the FVIII variants or functional fragments thereof. Examples of promoters that are active in liver are the TTR promoter, human alpha 1-antitrypsin (hAAT) promoter; albumin, Miyatake, et al. J. Virol., 71:5124-32 (1997); hepatitis B virus core promoter, Sandig, et al., Gene Ther. 3:1002-9 (1996); alpha-fetoprotein (AFP), Arbuthnot, et al., Hum. Gene. Ther., 7:1503-14 (1996)], among others. An example of an enhancer active in liver is apolipoprotein E (apoE) HCR-1 and HCR-2 (Allan et al., J. Biol. Chem., 272:29113-19 (1997)).

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al, Cell, 41:521-530 (1985)), the SV40 promoter, the dihydrofolate reductase promoter, the cytoplasmic β-actin promoter and the phosphoglycerol kinase (PGK) promoter.

Expression control elements also can confer expression in a manner that is regulatable, that is, a signal or stimuli increases or decreases expression of the operably linked heterologous polynucleotide. A regulatable element that increases expression of the operably linked polynucleotide in response to a signal or stimuli is also referred to as an “inducible element” (i.e., is induced by a signal). Particular examples include, but are not limited to, a hormone (e.g., steroid) inducible promoter. Typically, the amount of increase or decrease conferred by such elements is proportional to the amount of signal or stimuli present; the greater the amount of signal or stimuli, the greater the increase or decrease in expression. Particular non-limiting examples include zinc-inducible sheep metallothionine (MT) promoter; the steroid hormone-inducible mouse mammary tumor virus (MMTV) promoter; the T7 polymerase promoter system (WO 98/10088); the tetracycline-repressible system (Gossen, et al., Proc. Natl. Acad. Sci. USA, 89:5547-5551 (1992)); the tetracycline-inducible system (Gossen, et al., Science. 268:1766-1769 (1995); see also Harvey, et al., Curr. Opin. Chem. Biol. 2:512-518 (1998)); the RU486-inducible system (Wang, et al., Nat. Biotech. 15:239-243 (1997) and Wang, et al., Gene Ther. 4:432-441 (1997)]; and the rapamycin-inducible system (Magari, et al., J. Clin. Invest. 100:2865-2872 (1997); Rivera, et al., Nat. Medicine. 2:1028-1032 (1996)). Other regulatable control elements which may be useful in this context are those which are regulated by a specific physiological state, e.g., temperature, acute phase, development.

Expression control elements also include the native elements(s) for the heterologous polynucleotide, e.g., FVIII gene. A native control element (e.g., promoter) may be used when it is desired that expression of the heterologous polynucleotide should mimic the native expression. The native element may be used when expression of the heterologous polynucleotide is to be regulated temporally or developmentally, or in a tissue-specific manner, or in response to specific transcriptional stimuli. Other native expression control elements, such as introns, polyadenylation sites or Kozak consensus sequences may also be used.

The term “operably linked” means that the regulatory sequences necessary for expression of a coding sequence are placed in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (polyA) sequences) which flank a sequence, such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for AAV vector packaging into virus particle. In various embodiments, a filler/stuffer nucleic acid sequence is an untranslated (non-protein encoding) segment of nucleic acid. For a nucleic acid sequence less than 4.7 Kb, the filler or stuffer polynucleotide sequence has a length that when combined (e.g., inserted into a vector) with the sequence has a total length between about 3.0-5.5 Kb, or between about 4.0-5.0 Kb, or between about 4.3-4.8 Kb.

An intron can also function as a filler or stuffer polynucleotide sequence in order to achieve a length for AAV vector packaging into a virus particle. Introns and intron fragments that function as a filler or stuffer polynucleotide sequence also can enhance expression.

The phrase “hemostasis related disorder” refers to bleeding disorders such as hemophilia A, hemophilia A patients with inhibitory antibodies, deficiencies in coagulation Factors, VII, VIII, IX and X, XI, V, XII, II, von Willebrand factor, combined FV/FVIII deficiency, vitamin K epoxide reductase Cl deficiency, gamma-carboxylase deficiency; bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e. FXa inhibitors); and platelet disorders such as, Bernard Soulier syndrome, Glanzman thromblastemia, and storage pool deficiency.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane.

With respect to protein, the term “isolated protein” or “isolated and purified protein” is sometimes used herein. This term refers primarily to a protein produced by expression of a nucleic acid molecule. Alternatively, this term may refer to a protein which has been sufficiently separated from other proteins with which it would naturally be associated, so as to exist in “substantially pure” form.

The term “isolated” does not exclude combinations produced by the hand of man, for example, a recombinant vector (e.g., rAAV) sequence, or virus particle that packages or encapsidates a vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or about 90-99% by weight, of the compound of interest. Purity is measured by methods appropriate for the compound of interest (e.g. chromatographic methods, agarose or polyacrylamide gel electrophoresis, HPLC analysis, and the like).

The phrase “consisting essentially or when referring to a particular nucleotide sequence or amino acid sequence means a sequence having the properties of” a given sequence, e.g., FVIII. For example, when used in reference to an amino acid sequence, the phrase includes the sequence per se and molecular modifications that would not affect the basic and novel characteristics of the sequence.

The term “oligonucleotide,” as used herein refers to primers and probes, and is defined as a nucleic acid molecule comprised of two or more ribo- or deoxyribonucleotides, such as more than three. The exact size of the oligonucleotide will depend on various factors and on the particular application for which the oligonucleotide is used.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, for diagnostic applications, depending on the complexity of the target sequence, the oligonucleotide probe typically contains 15-25 or more nucleotides, although it may contain fewer nucleotides.

Probes can be selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to act functionally as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product.

The primer may vary in length depending on the particular conditions and requirements of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “identity,” “homology” and grammatical variations thereof, mean that two or more referenced entities are the same, when they are “aligned” sequences. Thus, by way of example, when two polypeptide sequences are identical, they have the same amino acid sequence, at least within the referenced region or portion. Where two polynucleotide sequences are identical, they have the same polynucleotide sequence, at least within the referenced region or portion. The identity can be over a defined area (region or domain) of the sequence. An “area” or “region” of identity refers to a portion of two or more referenced entities that are the same. Thus, where two protein or nucleic acid sequences are identical over one or more sequence areas or regions they share identity within that region. An “aligned” sequence refers to multiple polynucleotide or protein (amino acid) sequences, often containing corrections for missing or additional bases or amino acids (gaps) as compared to a reference sequence.

The identity can extend over the entire length or a portion of the sequence. In particular aspects, the length of the sequence sharing the percent identity is 2, 3, 4, 5 or more contiguous amino acids or nucleic acids, e.g., 6, 7, 8, 9, 10, 11, 12, 13, 14, 15, 16, 17, 18, 19, 20, etc. contiguous amino acids or nucleic acids. In additional particular aspects, the length of the sequence sharing identity is 21 or more contiguous amino acids or nucleic acids, e.g., 21, 22, 23, 24, 25, 26, 27, 28, 29, 30, 31, 32, 33, 34, 35, 36, 37, 38, 39, 40, etc. contiguous amino acids or nucleic acids. In further particular aspects, the length of the sequence sharing identity is 41 or more contiguous amino acids or nucleic acids, e.g. 42, 43, 44, 45, 45, 47, 48, 49, 50, etc., contiguous amino acids or nucleic acids. In yet further particular aspects, the length of the sequence sharing identity is 50 or more contiguous nucleic acids or amino acids, e.g., 50-55, 55-60, 60-65, 65-70, 70-75, 75-80, 80-85, 85-90, 90-95, 95-100, 100-110, etc. contiguous amino acids or nucleic acids.

As set forth herein, Factor VIII (FVIII) variants will be distinct from (e.g., non wild-type) but will exhibit sequence identity with wild-type FVIII. In FVIII variants, a variant will typically be at least about 70% identical, more typically about 75% identical, even more typically about 75-80%, 80-85%, 85-90%, 90-95% or 95-99%, 99-99.9% identical to wild-type FVIII. Thus, for example, a FVIII variant may have 95-99% or 99-99.9% identity to wild-type FVIII as set forth herein.

At the amino acid sequence level, a variant such as a FVIII variant protein will be at least about 70% identical, more typically about 80% identical, even more typically about 90% or more identity. In other embodiments, a variant such as a FVIII variant protein has at least 90%, 91%, 92%, 93%, 94%, 95%, 96%, 97%, 98%, 99%, 99.5%, 99.6%, 99.7%, 99.8%, 99.9%, or more identity to a reference sequence, e.g. wild-type FVIII protein with B-domain or having a B-domain deletion (FVIII-BDD). To determine identity, if the FVIII variant retains the B-domain, it is appropriate to compare identity to wild-type FVIII. If the FVIII variant has a B-domain deletion, it is appropriate to compare identity to wild-type FVIII that also has a B-domain deletion.

The terms “homologous” or “homology” mean that two or more referenced entities share at least partial identity over a given region or portion. “Areas, regions or domains” of homology or identity mean that a portion of two or more referenced entities share homology or are the same. Thus, where two sequences are identical over one or more sequence regions they share identity in these regions. “Substantial homology” means that a molecule is structurally or functionally conserved such that it has or is predicted to have at least partial structure or function of one or more of the structures or functions (e.g., a biological function or activity) of the reference molecule, or relevant/corresponding region or portion of the reference molecule to which it shares homology.

The extent of identity (homology) or “percent identity” between two sequences can be ascertained using a computer program and/or mathematical algorithm. For purposes of this invention comparisons of nucleic acid sequences are performed using the GCG Wisconsin Package version 9.1, available from the Genetics Computer Group in Madison, Wis. For convenience, the default parameters (gap creation penalty=12, gap extension penalty=4) specified by that program are intended for use herein to compare sequence identity. Alternately, the Blastn 2.0 program provided by the National Center for Biotechnology Information (found on the world wide web at ncbi.nlm.nih.gov/blast/; Altschul et al., 1990, J Mol Biol 215:403-410) using a gapped alignment with default parameters, may be used to determine the level of identity and similarity between nucleic acid sequences and amino acid sequences. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Nucleic acid molecules, expression vectors (e.g., vector genomes), plasmids, including nucleic acid encoding Factor VIII (FVIII) variants of the invention may be prepared by using recombinant DNA technology methods. The availability of nucleotide sequence information enables preparation of isolated nucleic acid molecules of the invention by a variety of means. For example, nucleic acid encoding Factor VIII (FVIII) variants can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Nucleic acids of the invention may be maintained as DNA in any convenient cloning vector. In a one embodiment, clones are maintained in a plasmid cloning/expression vector, such as pBluescript (Stratagene, La Jolla, Calif.), which is propagated in a suitable E. coli host cell. Alternatively, nucleic acids may be maintained in vector suitable for expression in mammalian cells. In cases where post-translational modification affects coagulation function, nucleic acid molecule can be expressed in mammalian cells.

Nucleic acid encoding Factor VIII (FVIII) variants of the invention include cDNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid of the invention. Such oligonucleotides are useful as probes for detecting FVIII expression.

A B-domain deleted, nucleic acid encoding FVIII variant of the invention, encoded by a nucleic acid variant, that has or encodes further modifications of a PACE/Furin cleavage site mutation, deletion or substitution, or a functional fragment of either as described herein, may be prepared in a variety of ways, according to known methods. The protein may be purified from appropriate sources, e.g., transformed bacterial or animal cultured cells or tissues which express engineered FVIII by immune-affinity purification.

The availability of invention FVIII variants and corresponding encoding nucleic acid molecules, which optionally also FVIII variant with or without a PACE/Furin cleavage site mutation, deletion or substitution, or a functional fragment of either as described herein, enables production of FVIII using in vitro expression methods known in the art. For example, a cDNA or gene may be cloned into an appropriate in vitro transcription vector, such as, but not limited to, pSP64 or pSP65 for in vitro transcription, followed by cell-free translation in a suitable cell-free translation system, such as wheat germ or rabbit reticulocyte lysates. In vitro transcription and translation systems are commercially available, e.g., from Promega Biotech, Madison, Wis. or BRL, Rockville, Md.

Alternatively, according to an embodiment, larger quantities of FVIII may be produced by expression in a suitable prokaryotic or eukaryotic expression system. For example, part or all of a nucleic acid encoding Factor VIII (FVIII) variant for example, may be inserted into a plasmid vector adapted for expression in a bacterial cell, such as E. coli or a mammalian cell line such as baby hamster kidney (BHK), CHO or Hela cells. Alternatively, in an embodiment, tagged fusion proteins comprising FVIII can be generated. Such FVIII-tagged fusion proteins are encoded by part or all of a DNA molecule, ligated in the correct codon reading frame to a nucleotide sequence encoding a portion or all of a desired polypeptide tag which is inserted into a plasmid vector adapted for expression in a bacterial cell, such as E. coli or a eukaryotic cell, such as, but not limited to, yeast and mammalian cells.

Vectors such as, but not limited to, those described herein comprise the regulatory elements necessary for expression of the DNA in the host cell positioned in such a manner as to permit expression of the encoded protein in the host cell. Such regulatory elements required for expression include, but are not limited to, promoter sequences, enhancer sequences and transcription initiation sequences as set forth herein and known to the skilled artisan.

Nucleic acid encoding Factor VIII (FVIII) variant optionally also encoding FVIII variant proteins as set forth herein (e.g., with a PACE/furin modification or deletion), produced by gene expression in a recombinant prokaryotic or eukaryotic system, may be purified according to methods known in the art. In an embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope, GST or the hemagglutinin epitope. Such methods are commonly used by skilled practitioners.

FVIII proteins, prepared by the aforementioned methods, may be analyzed according to standard procedures. For example, such proteins may be assessed for altered coagulation properties according to known methods.

As disclosed herein, a convenient way of producing a polypeptide according to the invention is to express nucleic acid encoding it, by use of the nucleic acid in an expression system. A variety of expression systems of utility for the methods of the invention are well known to those of skill in the art.

Accordingly, the invention also provides methods of making a FVIII variants (as disclosed), the method including expression of nucleic acid encoding FVIII variants. This may conveniently be achieved by culturing a host cell, containing such a vector, under appropriate conditions which cause or allow production of the polypeptide. Polypeptides may also be produced in in vitro systems.

Methods and uses of the invention of the invention include delivering (transducing) nucleic acid (transgene) into host cells, including dividing and/or non-dividing cells. The nucleic acids, recombinant vector (e.g., rAAV), methods, uses and pharmaceutical formulations of the invention are additionally useful in a method of delivering, administering or providing a protein to a subject in need thereof, as a method of treatment. In this manner, the nucleic acid is transcribed and the protein may be produced in vivo in a subject. The subject may benefit from or be in need of the protein because the subject has a deficiency of the protein, or because production of the protein in the subject may impart some therapeutic effect, as a method of treatment or otherwise.

Vectors including lenti- or parvo-virus vector (e.g., AAV) sequences, recombinant virus particles, methods and uses may be used to deliver a Factor VIII (FVIII) encoding nucleic acid variant with a biological effect to treat or ameliorate one or more symptoms associated with a FVIII deficiency or abnormality Recombinant lenti- or parvo-virus vector (e.g., AAV) sequences, plasmids, recombinant virus particles, methods and uses may be used to provide therapy for various disease states involving or due to a FVIII deficiency or abnormality.

Invention nucleic acids, vectors, recombinant vectors (e.g., rAAV), and recombinant virus particles, methods and uses permit the treatment of genetic diseases, e.g., a FVIII deficiency. For deficiency state diseases, gene transfer can be used to bring a normal gene into affected tissues for replacement therapy, as well as to create animal models for the disease using antisense mutations. For unbalanced disease states, gene transfer could be used to create a disease state in a model system, which could then be used in efforts to counteract the disease state. The use of site-specific integration of nucleic acid sequences to correct defects is also possible.

In particular embodiments, Factor VIII (FVIII) variants, or FVIII protein variants with a further modification such as, but not limited to, a PACE/Furin cleavage site mutation, deletion or substitution, or a functional fragment of either, may be used, for example, as therapeutic and/or prophylactic agents (protein or nucleic acid) which modulate the blood coagulation cascade or as a transgene in gene. For example, Factor VIII (FVIII) variants may have similar coagulation activity as wild-type FVIII, or altered coagulation activity compared to wild-type FVII, such as in the case of a FVIII protein variant (e.g., a FVIII with 1 or 2 mutations or deletions at amino acid positions 1657-58, or 1649-50, or 1439-40 or with PACE/Furin cleavage site mutation, deletion or substitution), or a functional fragment. Cell-based strategies allow continuous expression of such Factor VIII (FVIII) variants, for example, in a subject in need therefore such as hemophilia A patients. As disclosed herein, certain modifications of FVIII molecules (nucleic acid and protein) result in increased coagulation activity, increased expression at the nucleic acid level, and/or greater stability at the protein level thereby effectively improving hemostasis.

Factor VIII (FVIII) variants, optionally with a PACE/Furin cleavage site mutation, deletion or substitution, or a functional fragment of either may be used for a variety of purposes in accordance with the invention. In one embodiment, a nucleic acid delivery vehicle (i.e., an expression vector) is provided wherein the expression vector comprises a Factor VIII (FVIII) variant encoding nucleic acid, optionally with a PACE/Furin cleavage site mutation, deletion or substitution, or a functional fragment of either as described herein. Administration of FVIII variant-encoding expression vectors to a patient results in the expression of FVIII protein which serves to alter the coagulation cascade. In accordance with the invention, a nucleic acid encoding Factor VIII (FVIII) variant may also include a PACE/Furin cleavage site mutation, deletion or substitution, or a functional fragment, whose expression increases hemostasis. In one embodiment, a nucleic acid encoding Factor VIII (FVIII) variant encodes a FVIII polypeptide with a substitution or deletion at amino acid position(s) 1657-58, or 1649-50, or 1439-40. Such FVIII variants optionally have a PACE/Furin cleavage site mutation, deletion or substitution, or are a functional fragment of a FVIII variant.

In additional embodiments of the invention, compositions and methods are provided for administration of a viral vector comprising a nucleic acid encoding Factor VIII (FVIII) variants, FVIII protein variant with a PACE/Furin cleavage site mutation, deletion or substitution, or a functional fragment of either. In one embodiment, the expression vector nucleic acid encoding comprising Factor VIII (FVIII) variant without or with a PACE/Furin cleavage site mutation, deletion or substitution, or a functional fragment of either, is a viral vector.

Expression vectors comprising nucleic acid encoding Factor VIII (FVIII) variant, a nucleic acid encoding FVIII variant protein variant without or with a PACE/Furin cleavage site mutation, deletion or substitution, or a functional fragment of either, may be administered alone, or in combination with other molecules useful for modulating hemostasis. According to the invention, the expression vectors or combination of therapeutic agents may be administered to the patient alone or in a pharmaceutically acceptable or biologically compatible composition.

Viral vectors such as, but not limited to, lenti- and parvo-virus vectors, including AAV serotypes and variants thereof (e.g., pseudotype AAV) provide a means for delivery of nucleic acid into cells ex vivo, in vitro and in vivo, which encode proteins such that the cells express the encoded protein. AAV are viruses useful as gene therapy vectors as they can penetrate cells and introduce nucleic acid/genetic material so that the nucleic acid/genetic material may be stably maintained in cells. In addition, such viruses can introduce nucleic acid/genetic material into specific sites, for example. Because AAV are not associated with pathogenic disease in humans, AAV vectors are able to deliver heterologous polynucleotide sequences (e.g., therapeutic proteins and agents) to human patients without causing substantial AAV pathogenesis or disease.

Viral vectors which may be used in the invention include, but are not limited to, adeno-associated virus (AAV) vectors of multiple serotypes (e.g., AAV-1 to AAV-12, and others) and hybrid/chimeric AAV vectors, lentivirus vectors and pseudo-typed lentivirus vectors (e.g., Ebola virus, vesicular stomatitis virus (VSV), and feline immunodeficiency virus (FIV)), herpes simplex virus vectors, adenoviral vectors (with or without tissue specific promoters/enhancers), vaccinia virus vectors, retroviral vectors, lentiviral vectors, non-viral vectors and others.

AAV and lentiviral particles may be used as vehicles for effective gene delivery. Such virions possess a number of desirable features for such applications, including tropism for dividing and non-dividing cells. Early clinical experience with these vectors also demonstrated no sustained toxicity and immune responses were minimal or undetectable. AAV are known to infect a wide variety of cell types in vivo and in vitro by receptor-mediated endocytosis or by transcytosis. These vector systems have been tested in humans targeting retinal epithelium, liver, skeletal muscle, airways, brain, joints and hematopoietic stem cells. Non-viral vectors, for example, based on plasmid DNA or minicircles, are also suitable gene transfer vectors for a large gene as that encoding FVIII.

It may be desirable to introduce a vector that can provide, for example, multiple copies of a desired gene and hence greater amounts of the product of that gene. Improved AAV and lentiviral vectors and methods for producing these vectors have been described in detail in a number of publications, patents, and patent applications, including: Wright J. F. (Hum Gene Ther 20:698-706, 2009). Lentiviral vector can also be produced and other vectors are available through the Lentivirus vector production core laboratory by NHLBI Gene Therapy Resource Program (GTRP)-Lentivirus Vector Production Core Laboratory.

In various embodiments of the invention a vector includes a lenti- or parvo-viral vector, such as an adeno-viral vector. In particular embodiments, a recombinant vector is a parvovirus vector. Parvoviruses are small viruses with a single-stranded DNA genome. “Adeno-associated viruses” (AAV) are in the parvovirus family.

Accordingly, the invention provides viral vectors that include nucleic acid encoding Factor VIII (FVIII) variants, optionally such FVIII variants also with a PACE/Furin cleavage site mutation, deletion or substitution, or a functional fragment of either. For example, a recombinant AAV vector can include a nucleic acid encoding Factor VIII, such as, but not limited to, a Factor VIII (FVIII) variant, where the encoded FVIII protein optionally has B-domain deletion, and/or where the encoded FVIII protein optionally has a PACE/Furin cleavage site mutation, deletion or substitution, or a functional fragment of either. Vector delivery or administration to a subject (e.g., mammal) therefore provides FVIII variant protein, without or with a PACE/Furin cleavage site mutation, deletion or substitution, or a functional fragment of either, to a subject such as a mammal (e.g., human) that would benefit from or is in need thereof.

Direct delivery of vectors or ex-vivo transduction of human cells followed by infusion into the body will result in FVIII expression thereby exerting a beneficial therapeutic effect on hemostasis. In the context of invention Factor VIII described herein, such administration enhances pro-coagulation activity.

AAV vectors and lentiviral vectors do not typically include viral genes associated with pathogenesis. Such vectors typically have one or more of the wild type AAV genes deleted in whole or in part, such as rep and/or cap genes, but retain at least one functional flanking ITR sequence, as necessary for the rescue, replication, and packaging of the recombinant vector into an AAV vector particle. For example, only the essential parts of vector e.g., the ITR and LTR elements, respectively are included. An AAV vector genome would therefore include sequences required in cis for replication and packaging (e.g., functional ITR sequences)

Recombinant AAV vector, as well as methods and uses thereof, include any viral strain or serotype. As a non-limiting example, a recombinant AAV vector can be based upon any AAV genome, such as AAV-1, -2, -3, -4, -5, -6, -7, -8, -9, -10, -11, -12, -rh74, -rh10 or AAV-2i8, for example. Such vectors can be based on the same strain or serotype (or subgroup or variant), or be different from each other. As a non-limiting example, a recombinant AAV vector based upon one serotype genome can be identical to one or more of the capsid proteins that package the vector. In addition, a recombinant AAV vector genome can be based upon an AAV (e.g., AAV2) serotype genome distinct from one or more of the AAV capsid proteins that package the vector. For example, the AAV vector genome can be based upon AAV2 or AAV6, whereas at least one of the three capsid proteins could be a AAV1, AAV3, AAV4, AAV5, AAV6, AAV7, AAV8, AAV9, AAV10, AAV11, AAV12, Rh10, Rh74 or AAV-2i8 or variant thereof, for example.

In particular embodiments, adeno-associated virus (AAV) vectors include AAV1, AAV2, AAV3, AAV4, AAV5, AAV6, AAV7, AAV8, AAV9, AAV10, AAV11, AAV12, Rh10, Rh74 and AAV-2i8, as well as variants (e.g., capsid variants, such as amino acid insertions, additions and substitutions) thereof as set forth in WO 2013/158879 (International Application PCT/US2013/037170) and WO 2015/013313 (International Application PCT/US2014/047670). AAV variants include AAV1, AAV2, AAV3, AAV4, AAV5, AAV6, AAV7, AAV8, AAV9, AAV10, AAV11, AAV12, Rh10, Rh74 and AAV-2i8 variants. Accordingly, AAV vectors and AAV variants (e.g., capsid variants) that include (encapsidate or package) nucleic acid encoding Factor VIII (FVIII) variants without or with a PACE/Furin cleavage site mutation, deletion or substitution, or a functional fragment of either, are provided.

AAV and AAV variants (e.g., capsid variants) serotypes (e.g., VP1, VP2, and/or VP3 sequences) may or may not be distinct from other AAV serotypes, including, for example, AAV1-AAV12, Rh74 or or Rh10 (e.g., distinct from VP1, VP2, and/or VP3 sequences of any of AAV1-AAV12, Rh74 or Rh10 serotypes).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates of are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

AAV vectors therefore include gene/protein sequences identical to gene/protein sequences characteristic for a particular serotype. As used herein, an “AAV vector related to AAV1” refers to one or more AAV proteins (e.g., VP1, VP2, and/or VP3 sequences) that has substantial sequence identity to one or more polynucleotides or polypeptide sequences that comprise AAV1. Analogously, an “AAV vector related to AAV8” refers to one or more AAV proteins (e.g., VP1, VP2, and/or VP3 sequences) that has substantial sequence identity to one or more polynucleotides or polypeptide sequences that comprise AAV8. An “AAV vector related to AAV-Rh74” refers to one or more AAV proteins (e.g., VP1, VP2, and/or VP3 sequences) that has substantial sequence identity to one or more polynucleotides or polypeptide sequences that comprise AAV-Rh74. Such AAV vectors related to another serotype, e.g., AAV1, AAV2, AAV3, AAV4, AAV5, AAV6, AAV7, AAV8, AAV9, AAV10, AAV11, AAV12, Rh10, Rh74 or AAV-2i8, can therefore have one or more distinct sequences from AAV1, AAV2, AAV3, AAV4, AAV5, AAV6, AAV7, AAV8, AAV9, AAV10, AAV11, AAV12, Rh10, Rh74 and AAV-2i8, but can exhibit substantial sequence identity to one or more genes and/or proteins, and/or have one or more functional characteristics of AAV1, AAV2, AAV3, AAV4, AAV5, AAV6, AAV7, AAV8, AAV9, AAV10, AAV11, AAV12, Rh10, Rh74 or AAV-2i8 (e.g., such as cell/tissue tropism). Exemplary non-limiting AAV variants include capsid variants of any of VP1, VP2, and/or VP3.

In various exemplary embodiments, an AAV vector related to a reference serotype has a polynucleotide, polypeptide or subsequence thereof that includes or consists of a sequence at least 80% or more (e.g., 85%, 90%, 95%, 96%, 97%, 98%, 99%, 99.1%, 99.2%, 99.3%, 99.4%, 99.5%, etc.) identical to one or more AAV1, AAV2, AAV3, AAV4, AAV5, AAV6, AAV7, AAV8, AAV9, AAV10, AAV11, AAV12, Rh10, Rh74 or AAV-2i8 (e.g., such as VP1, VP2, and/or VP3 sequences).

AAV ITR(s) may be the same serotype as AAV capsid, or of a different serotype. For example a capsid may be AAV8 and the ITR(s) is AAV2 or AAV6.

Compositions, methods and uses of the invention include AAV sequences (polypeptides and nucleotides), and subsequences thereof that exhibit less than 100% sequence identity to a reference AAV serotype such as, but not limited to, AAV1, AAV2, AAV3, AAV4, AAV5, AAV6, AAV7, AAV8, AAV9, AAV10, AAV11, AAV12, Rh10, or AAV-2i8, but are distinct from and not identical to known AAV genes or proteins, such as, but not limited to, AAV1, AAV2, AAV3, AAV4, AAV5, AAV6, AAV7, AAV8, AAV9, AAV10, AAV11, AAV12, Rh10, Rh74 or AAV-2i8, genes or proteins, etc. In one embodiment, an AAV polypeptide or subsequence thereof includes or consists of a sequence at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to any reference AAV sequence or subsequence thereof, such as, but not limited to, AAV1, AAV2, AAV3, AAV4, AAV5, AAV6, AAV7, AAV8, AAV9, AAV10, AAV11, AAV12, Rh10, Rh74 or AAV-2i8 (e.g., VP1, VP2 and/or VP3). In particular aspects, an AAV variant has 1, 2, 3, 4, 5, 5-10, 10-15, 15-20, 20-30, 30-50, or more amino acid substitutions.

Recombinant AAV vectors, including AAV1, AAV2, AAV3, AAV4, AAV5, AAV6, AAV7, AAV8, AAV9, AAV10, AAV11, AAV12, Rh10, Rh74 or AAV-2i8 and variant, related, hybrid and chimeric sequences, can be constructed using recombinant techniques that are known to the skilled artisan, to include one or more nucleic acid sequences (transgenes) flanked with one or more functional AAV ITR sequences.

In one embodiment of the invention, FVIII polypeptide variants, such as, but not limited to, a human FVIII variant with 1 or 2 mutations or deletions at amino acid position(s) 1657-58, or 1649-50, or 1439-40, optionally with a PACE-furin cleavage recognition site mutation, deletion or substitution, may be administered to a patient via infusion in a biologically compatible carrier, for example, via intravenous injection. The FVIII polypeptide variants, such as, but not limited to, a human FVIII variant with 1 or 2 mutations or deletions at amino acid position(s) 1657-58, or 1649-50, or 1439-40, optionally with a PACE-furin cleavage recognition site mutation, deletion or substitution of the invention may optionally be encapsulated into liposomes or mixed with other phospholipids or micelles to increase stability of the molecule. FVIII protein with 1 or 2 mutations or deletions at amino acid position(s) 1657-58, or 1649-50, or 1439-40, optionally with a PACE/Furin cleavage site mutation, deletion or substitution, or a functional fragment, may be administered alone or in combination with other agents known to modulate hemostasis (e.g., Factor V, Factor Va or derivatives thereof).

An appropriate composition in which to deliver FVIII polypeptide variants, such as, but not limited to, a human FVIII variant with 1 or 2 mutations or deletions at amino acid position(s) 1657-58, or 1649-50, or 1439-40, optionally with a PACE-furin cleavage recognition site mutation, deletion or substitution may be determined by a medical practitioner upon consideration of a variety of physiological variables, including, but not limited to, the patient's condition and hemodynamic state. A variety of compositions well suited for different applications and routes of administration are well known in the art and are described hereinbelow.

A preparation containing purified FVIII protein, such as, but not limited to, FVIII variant with 1 or 2 mutations or deletions at amino acid position(s) 1657-58, or 1649-50, or 1439-40, optionally with a PACE/Furin cleavage site mutation, deletion or substitution, or a functional fragment, contains a physiologically acceptable matrix and may be formulated as a pharmaceutical preparation. The preparation can be formulated using substantially known prior art methods, it can be mixed with a buffer containing salts, such as NaCl, CaCl₂, and amino acids, such as glycine and/or lysine, and in a pH range from 6 to 8. Until needed, the purified preparation containing FVIII variant can be stored in the form of a finished solution or in lyophilized or deep-frozen form.

A preparation can be stored in lyophilized form and is dissolved into a visually clear solution using an appropriate reconstitution solution. Alternatively, the preparation according to the invention can also be made available as a liquid preparation or as a liquid that is deep-frozen. The preparation according to the invention may optionally be especially stable, i.e., it can be allowed to stand in dissolved form for a prolonged time prior to administration or delivery.

The preparation according to the invention can be made available as a pharmaceutical preparation with FVIII activity in the form of a one-component preparation or in combination with other factors in the form of a multi-component preparation. Prior to processing the purified protein into a pharmaceutical preparation, the purified protein is subjected to the conventional quality controls and fashioned into a therapeutic form of presentation. In particular, during the recombinant manufacture, the purified preparation is tested for the absence of cellular nucleic acids as well as nucleic acids that are derived from the expression vector, such as is described in EP 0 714 987.

The pharmaceutical protein preparation may be used at dosages of between 30-100 IU/kg (One I.U is 100 ng/ml) at as single daily injection or up to 3 times/day for several days. Patients may be treated immediately upon presentation at the clinic with a bleed. Alternatively, patients may receive a bolus infusion every eight to twelve hours, or if sufficient improvement is observed, a once daily infusion of the FVIII variant described herein.

The invention also provides compositions, such as, but not limited to, compositions including Factor VIII (FVIII) variants as set forth herein, such as FVIII variant with 1 or 2 mutations or deletions at amino acid position(s) 1657-58, or 1649-50, or 1439-40, optionally with a PACE/Furin cleavage site mutation, deletion or substitution, and functional fragments of either, having one or more of the following attributes: 1) exhibits increased expression by cells or in animals; 2) exhibits increased secretion by cells; 3) exhibits increased activity, as reflected by increased clotting, for example; 4) exhibits increased stability; and/or 5) exhibits increased packaging by AAV vector.

Accordingly, invention FVIII variants, nucleic acids, vectors, recombinant vectors (e.g., rAAV), and recombinant virus particles and other compositions, agents, drugs, biologics (proteins) can be incorporated into pharmaceutical compositions. Such pharmaceutical compositions are useful for, among other things, administration and delivery to a subject in vivo or ex vivo.

In particular embodiments, pharmaceutical compositions also contain a pharmaceutically acceptable carrier or excipient. Such excipients include any pharmaceutical agent that does not itself induce an immune response harmful to the individual receiving the composition, and which may be administered without undue toxicity.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a protein, nucleic acid, vector, viral particle or protein to a subject.

Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, a preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

Pharmaceutical compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran.

Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. For administration of FVIII variant-containing vectors or polypeptides, such labeling would include amount, frequency, and method of administration.

Pharmaceutical compositions and delivery systems appropriate for the compositions, methods and uses of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, NJ; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

The invention also provides methods for introducing Factor VIII (FVIII) variants, nucleic acid encoding FVIII variants, such as, but not limited to, a FVIII variant as set forth herein such as FVIII with 1 or 2 mutations or deletions at amino acid position(s) 1657-58, or 1649-50, or 1439-40, optionally with a PACE/Furin cleavage site mutation, deletion or substitution, and functional fragments of either, into an animal or a cell. In one embodiment, a method includes contact or administration of an individual (patient or subject such as a mammal) with a FVIII variant, such as, but not limited to, FVIII variant with 1 or 2 mutations or deletions at amino acid position(s) 1657-58, or 1649-50, or 1439-40, optionally with a PACE/Furin cleavage site mutation, deletion or substitution, or a functional fragment of either.

In another embodiment, a method includes contact or administration of an individual (patient or subject such as a mammal) with a nucleic acid delivery vehicle (e.g., an AAV vector) comprising a nucleic acid encoding Factor VIII (FVIII) variant, such as, but not limited to, FVIII variant with 1 or 2 mutations or deletions at amino acid position(s) 1657-58, or 1649-50, or 1439-40, optionally with a PACE/Furin cleavage site mutation, deletion or substitution, or a functional fragment of either, under conditions wherein the FVIII polypeptide is expressed in the individual. In another embodiment, a method includes providing cells of an individual (patient or subject such as a mammal) with a nucleic acid delivery vehicle (e.g., an AAV vector) comprising a nucleic acid encoding Factor VIII (FVIII) variant as set forth herein such as, but not limited to, a FVIII with 1 or 2 mutations or deletions at amino acid position(s) 1657-58, or 1649-50, or 1439-40, optionally with a PACE/Furin cleavage site mutation, deletion or substitution, or a functional fragment of either, under conditions wherein the FVIII polypeptide is expressed in the individual. In a particular aspect, a method is for modulating hemostasis.

From the foregoing, it can be seen that Factor VIII variants or nucleic acid encoding Factor VIII (FVIII) variants set forth herein such as, but not limited to, FVIII variant with 1 or 2 mutations or deletions at amino acid position(s) 1657-58, or 1649-50, or 1439-40, optionally with a PACE/Furin cleavage site mutation, deletion or substitution, or a functional fragment of either, may be used in the treatment of disorders associated with deficient, insufficient or aberrant blood coagulation.

Compositions of Factor VIII variants or nucleic acid encoding Factor VIII (FVIII) variants as set forth herein such as, but not limited to, FVIII variant with 1 or 2 mutations or deletions at amino acid position(s) 1657-58, or 1649-50, or 1439-40, optionally with a PACE/Furin cleavage site mutation, deletion or substitution, or functional fragments, of either, including vectors, recombinant vectors (e.g., rAAV), and recombinant virus particles can be administered, and methods and uses of the invention can be provided, in a sufficient or effective amount to a subject in need thereof.

An “effective amount” or “sufficient amount” refers to an amount that provides, in single or multiple doses, alone or in combination, with one or more other compositions (therapeutic agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured). The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The dose to achieve a therapeutic effect, e.g., the dose in vector genomes/per kilogram of body weight (vg/kg), will vary based on several factors including, but not limited to: route of administration, the level of heterologous polynucleotide expression required to achieve a therapeutic effect, the specific disease treated, any host immune response to the viral vector, a host immune response to the heterologous polynucleotide or expression product (protein), and the stability of the protein expressed. One skilled in the art can determine a rAAV/vector genome dose range to treat a patient having a particular disease or disorder based on the aforementioned factors, as well as other factors. Generally, doses will range from at least 1×10⁸, or more, for example, 1×10⁹, 1×10¹⁰, 1×10¹¹, 1×10¹², 1×10¹³ or 1×10¹⁴, or more, vector genomes per kilogram (vg/kg) of the weight of the subject, to achieve a therapeutic effect. AAV dose in the range of 1×10¹⁰-1×10¹¹ in mice, and 1×10¹²-1×10¹³ in dogs have been effective.

Using hemophilia B as an example, generally speaking, it is believed that, in order to achieve a therapeutic effect, a blood coagulation factor concentration that is greater than 1% of factor concentration found in a normal individual is needed to change a severe disease phenotype to a moderate one. A severe phenotype is characterized by joint damage and life-threatening bleeds. To convert a moderate disease phenotype into a mild one, it is believed that a blood coagulation factor concentration greater than 5% of normal is needed. With respect to treating such a hemophilic subject, a typical dose is at least 1×10¹⁰ vector genomes (vg) per kilogram (vg/kg) of the weight of the subject, or between about 1×10¹⁰ to 1×10¹¹vg/kg of the weight of the subject, or between about 1×10¹¹ to 1×10¹²vg/kg of the weight of the subject, or between about 1×10¹² to 1×10¹³ vg/kg of the weight of the subject, to achieve a desired therapeutic effect.

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol, such as administration of recombinant clotting factor protein (e.g., FVIII) for treatment of a clotting disorder (e.g., hemophilia A).

Accordingly, methods and uses of the invention also include, among other things, methods and uses that result in a reduced need or use of another compound, agent, drug, therapeutic regimen, treatment protocol, process, or remedy. For example, for a blood clotting disease, a method or use of the invention has a therapeutic benefit if in a given subject a less frequent or reduced dose or elimination of administration of a recombinant clotting factor protein to supplement for the deficient or defective (abnormal or mutant) endogenous clotting factor in the subject. Thus, in accordance with the invention, methods and uses of reducing need or use of another treatment or therapy are provided.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

The term “ameliorate” means a detectable or measurable improvement in a subject's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease. For HemA, an effective amount would be an amount that reduces frequency or severity of acute bleeding episodes in a subject, for example, or an amount that reduces clotting time as measured by a clotting assay, for example.

Accordingly, pharmaceutical compositions of the invention include compositions wherein the active ingredients are contained in an effective amount to achieve the intended therapeutic purpose. Determining a therapeutically effective dose is well within the capability of a skilled medical practitioner using the techniques and guidance provided in the invention.

Therapeutic doses will depend on, among other factors, the age and general condition of the subject, the severity of the aberrant blood coagulation phenotype, the FVIII variant used, such as a FVIII variant set forth herein, such as, but not limited to, FVIII with 1 or 2 mutations or deletions at amino acid position(s) 1657-58, or 1649-50, or 1439-40, optionally with a PACE/Furin cleavage site mutation, deletion or substitution, or a functional fragment of either, and/or the strength of the control sequences regulating the expression levels of nucleic acid encoding Factor VIII (FVIII) variant. Thus, a therapeutically effective amount in humans will fall in a relatively broad range that may be determined by a medical practitioner based on the response of an individual patient to vector-based FVIII treatment.

Compositions such as pharmaceutical compositions may be delivered to a subject, so as to allow introduction or production of a biologically active protein (e.g., Factor VIII variant or nucleic acid encoding Factor VIII (FVIII) variant as set forth herein, such as, but not limited to, FVIII variant with 1 or 2 mutations or deletions at amino acid position(s) 1657-58, or 1649-50, or 1439-40, optionally with a PACE/Furin cleavage site mutation, deletion or substitution, or a functional fragment of either, or by inducing continuous expression of the FVIII transgene in vivo by gene- and or cell-based therapies or by ex-vivo modification of the patient's or donor's cells. In a particular embodiment, pharmaceutical compositions comprising sufficient genetic material to enable a recipient to produce a therapeutically effective amount of a FVIII polypeptide can influence hemostasis in the subject. Alternatively, as disclosed herein, an effective amount of a Factor VIII variant polypeptide as set forth herein, such as, but not limited to, FVIII variant with 1 or 2 mutations or deletions at amino acid position(s) 1657-58, or 1649-50, or 1439-40, optionally with a PACE/Furin cleavage site mutation, deletion or substitution, or a functional fragment of either, may be directly infused into a patient in need thereof.

The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents (e.g., co-factors) which influence hemostasis.

Factor VIII variant polypeptides and/or nucleic acid encoding FVIII variant used alone, or in combination with other agents, may be administered or contacted or directly infused into a patient in an appropriate biological carrier as described herein. Expression vectors of the invention comprising nucleic acid sequences encoding FVIII variant as set forth herein, such as, but not limited to, FVIII variant with 1 or 2 mutations or deletions at amino acid position(s) 1657-58, optionally with a PACE/Furin cleavage site mutation, deletion or substitution, or a functional fragment of either, may be administered to a patient by a variety of means to achieve and optionally maintain for a period of time a prophylactically and/or therapeutically effective level of FVIII polypeptide. One of skill in the art could readily determine specific protocols for using the FVIII encoding expression vectors of the invention for the therapeutic treatment of a particular patient.

Protocols for the generation of adenoviral vectors and administration to patients have been described in U.S. Pat. Nos. 5,998,205; 6,228,646; 6,093,699; 6,100,242; and International Patent Application Nos. WO 94/17810 and WO 94/23744, which are incorporated herein by reference in their entirety. In particular, for example, AAV vectors are employed to deliver nucleic acid encoded Factor VIII (FVIII) variant as set forth herein, such as, but not limited to, FVIII variant with 1 or 2 mutations or deletions at amino acid position(s) 1657-58, optionally with a PACE/Furin cleavage site mutation, deletion or substitution, or a functional fragment of either, to a patient in need thereof.

Factor VIII (FVIII) variants as set forth herein, such as, but not limited to, FVIII variant with 1 or 2 mutations or deletions at amino acid position(s) 1657-58, optionally with a PACE/Furin cleavage site mutation, deletion or substitution, or a functional fragment of either, and nucleic acid encoding FVIII variants as set forth herein, such as, but not limited to, FVIII variant with 1 or 2 mutations or deletions at amino acid position(s) 1657-58, optionally with a PACE/Furin cleavage site mutation, deletion or substitution, or a functional fragment of either, delivered by way of AAV vectors of the invention may be administered to a patient by any means known. Methods and uses of the invention include delivery and administration systemically, regionally or locally, or by any route, for example, by injection or infusion. Delivery of the pharmaceutical compositions in vivo may generally be accomplished via injection using a conventional syringe, although other delivery methods such as convection-enhanced delivery are envisioned (See e.g., U.S. Pat. No. 5,720,720). For example, compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. Other modes of administration include oral and pulmonary administration, suppositories, and transdermal applications. A clinician specializing in the treatment of patients with blood coagulation disorders may determine the optimal route for administration of the adenoviral-associated vectors comprising nucleic acid encoding FVIII variants based on a number of criteria, including, but not limited to: the condition of the patient and the purpose of the treatment (e.g., enhanced or reduced blood coagulation).

Invention methods and uses can be combined with any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents and drugs. Such biologics (proteins), agents, drugs, treatments and therapies can be administered or performed prior to, substantially contemporaneously with or following any other method or use of the invention, for example, a therapeutic method of treating a subject for a blood clotting disease such as HemA.

The compound, agent, drug, treatment or other therapeutic regimen or protocol can be administered as a combination composition, or administered separately, such as concurrently or in series or sequentially (prior to or following) delivery or administration of a nucleic acid, vector, recombinant vector (e.g., rAAV), or recombinant virus particle. The invention therefore provides combinations in which a method or use of the invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a nucleic acid, vector, recombinant vector (e.g., rAAV), or recombinant virus particle of the invention, to a subject.

The invention is useful in animals including human and veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of blood clotting diseases such as HemA and others known to those of skill in the art.

Subjects appropriate for treatment in accordance with the invention include those having or at risk of producing an insufficient amount or having a deficiency in a functional gene product (e.g., FVIII protein), or produce an aberrant, partially functional or non-functional gene product (e.g., FVIII protein), which can lead to disease. Subjects appropriate for treatment in accordance with the invention also include those having or at risk of producing an aberrant, or defective (mutant) gene product (protein) that leads to a disease such that reducing amounts, expression or function of the aberrant, or defective (mutant) gene product (protein) would lead to treatment of the disease, or reduce one or more symptoms or ameliorate the disease. Target subjects therefore include subjects having aberrant, insufficient or absent blood clotting factor production, such as hemophiliacs (e.g., hemophilia A).

Subjects appropriate for treatment in accordance with the invention also include those having or at risk of producing antibodies against AAV. AAV vectors can be administered or delivered to such subjects using several techniques. For example, empty capsid AAV (i.e., AAV lacking a FVIII nucleic acid) can be delivered to bind to the AAV antibodies in the subject thereby allowing the AAV vector bearing the FVIII nucleic acid to transform cells of the subject. Amounts of empty capsid AAV to administer can be calibrated based upon the amount of AAV antibodies produced in a particular subject. Empty capsid can be of any AAV serotype, for example, AAV1, AAV2, AAV3, AAV4, AAV5, AAV6, AAV7, AAV8, AAV9, AAV10, AAV11, AAV12, Rh10, Rh74 or AAV-2i8.

Alternatively or in addition to, AAV vector can be delivered by direct intramuscular injection (e.g., one or more slow-twitch fibers of a muscle). In another alternative, a catheter introduced into the femoral artery can be used to delivery AAV vectors to liver via the hepatic artery. Non-surgical means can also be employed, such as endoscopic retrograde cholangiopancreatography (ERCP), to deliver AAV vectors directly to the liver, thereby bypassing the bloodstream and AAV antibodies. Other ductal systems, such as the ducts of the submandibular gland, can also be used as portals for delivering AAV vectors into a subject that develops or has preexisting anti-AAV antibodies.

Administration or in vivo delivery to a subject can be performed prior to development of an adverse symptom, condition, complication, etc. caused by or associated with the disease. For example, a screen (e.g., genetic) can be used to identify such subjects as candidates for invention compositions, methods and uses. Such subjects therefore include those screened positive for an insufficient amount or a deficiency in a functional gene product (e.g., FVIII protein), or that produce an aberrant, partially functional or non-functional gene product (e.g., FVIII protein).

Administration or in vivo delivery to a subject in accordance with the methods and uses of the invention as disclosed herein can be practiced within 1-2, 2-4, 4-12, 12-24 or 24-72 hours after a subject has been identified as having the disease targeted for treatment, has one or more symptoms of the disease, or has been screened and is identified as positive as set forth herein even though the subject does not have one or more symptoms of the disease. Of course, methods and uses of the invention can be practiced 1-7, 7-14, 14-21, 21-48 or more days, months or years after a subject has been identified as having the disease targeted for treatment, has one or more symptoms of the disease, or has been screened and is identified as positive as set forth herein.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Recombinant vector (e.g., rAAV) sequences, recombinant virus particles, and pharmaceutical compositions thereof can be packaged in single or multiple unit dosage form for ease of administration and uniformity of dosage.

The invention provides kits with packaging material and one or more components therein. A kit typically includes a label or packaging insert including a description of the components or instructions for use in vitro, in vivo, or ex vivo, of the components therein. A kit can contain a collection of such components, e.g., a FVIII variant, nucleic acid, recombinant vector, virus (e.g., AAV) vector, or virus particle and optionally a second active, such as another compound, agent, drug or composition.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All patents, patent applications, publications, and other references, GenBank citations and ATCC citations cited herein are incorporated by reference in their entirety. In case of conflict, the specification, including definitions, will control.

Various terms relating to the biological molecules of the invention are used hereinabove and also throughout the specification and claims.

All of the features disclosed herein may be combined in any combination. Each feature disclosed in the specification may be replaced by an alternative feature serving a same, equivalent, or similar purpose. Thus, unless expressly stated otherwise, disclosed features (e.g., FVIII variant, nucleic acid, vector, plasmid, recombinant vector (e.g., rAAV) sequence, or recombinant virus particle) are an example of a genus of equivalent or similar features.

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such viruses/particles.

As used herein, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to at least 70% or more includes 70, 71, 72, etc. all the way up to the number 100.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-850, includes ranges of 1-20, 1-30, 1-40, 1-50, 1-60, 10-30, 10-40, 10-50, 10-60, 10-70, 10-80, 20-40, 20-50, 20-60, 20-70, 20-80, 20-90, 50-75, 50-100, 50-150, 50-200, 50-250, 100-200, 100-250, 100-300, 100-350, 100-400, 100-500, 150-250, 150-300, 150-350, 150-400, 150-450, 150-500, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### Example 1

### Generation and Delivery of AAV Vectors Expressing FVIII a3 Variants.

PACE-furin is not a candidate protease for the cleavage at position 1657-58 based on the amino acid sequence at this site. Since the protease that cleaves this site is not known, the residues that are critical for the cleavage are not known. Thus, modification of the residue at 1657, 1658 or at both positions (termed a3 variants) may result in altered cleavage at this site. Additional amino acid residues at this site may also be important for recognition and cleavage.

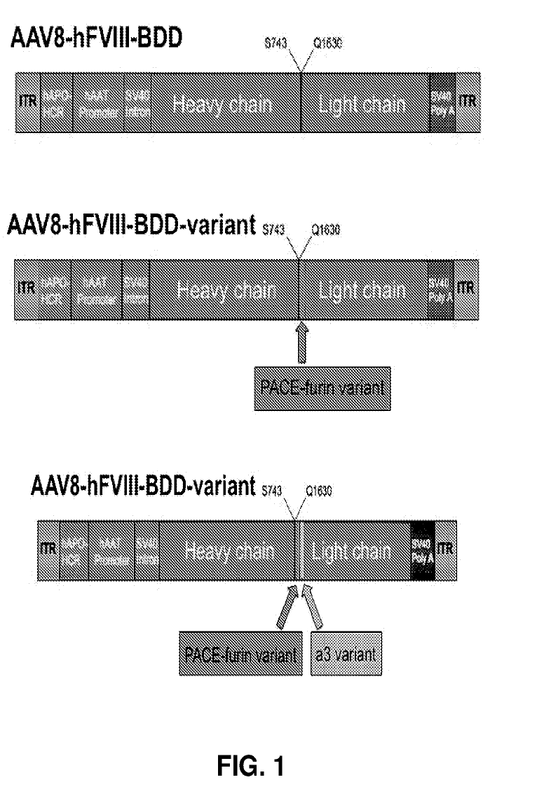

To determine substituting the hFVIII amino acid at position 1657-58 or deleting that site may increase the amount of single chain material and/or increase the procoagulant activity and/or increase secretion of the hFVIII, modifications were made at position 1657-58 in a hFVIII expression cassette (FIG. 1). The residues at position 1657-58 were replaced with amino acids to generate 51657P and D1658E variants. These constructs utilize a hAAT promoter and the wild type B-domain deleted human FVIII cDNA that is not codon-optimized. The difference between these constructs is the PACE-furin 43 deletion (del1645-47) that is the best performing PACE-furin deletion and the a3 modification (FIG. 1). In previous work we have introduced the PACE-furin variants into the wild type hFVIII sequence (non-codon-optimized) as well as codon-optimized versions and observed that the variants increased FVIII expression 2-3 fold.

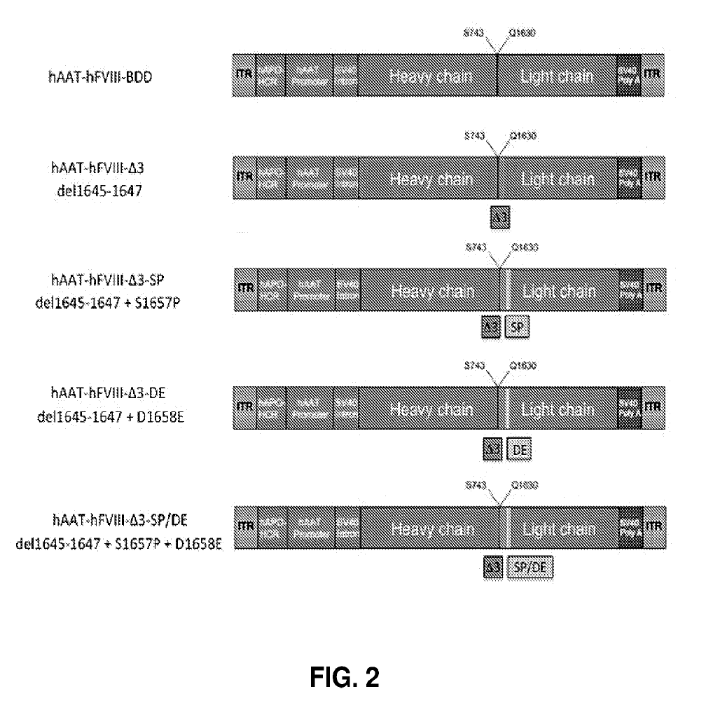

Five hFVIII constructs were compared in this study. FIG. 2 shows the: (1) human FVIII; (2) human FVIII with the Δ3 PACE-furin deletion; (3) human FVIII with the Δ3 PACE-furin deletion and the S1657P modification; (4) human FVIII with the Δ3 PACE-furin deletion and the D1658E modification; and (5) human FVIII with the Δ3 PACE-furin deletion and both the S1657P and D1658E modifications.

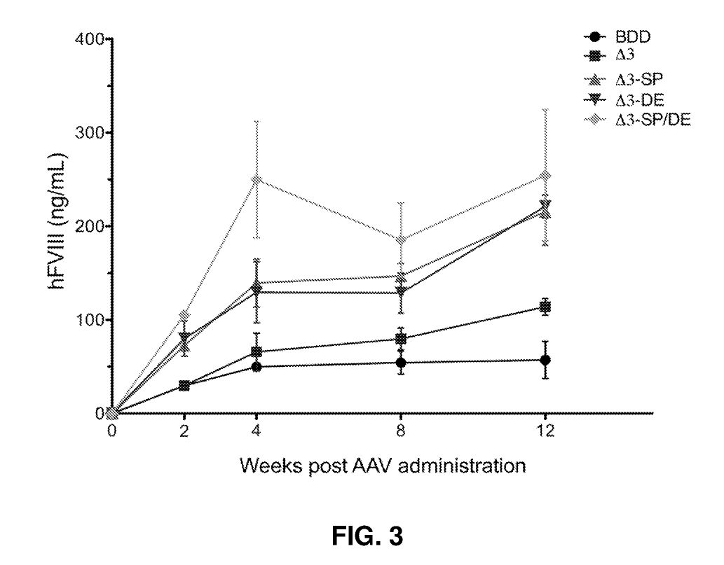

AAV8 vectors were generated with these 5 constructs and the vectors were titered side by side by quantitative PCR and silver staining. Hemophilia A/CD4 knockout (HA/CD4KO) mice were administered 5×10e11vg/mouse and plasma samples were collected at 2, 4 and 8 weeks post vector administration. A hFVIII specific ELISA was performed to determine the antigen levels after AAV delivery (FIG. 3).

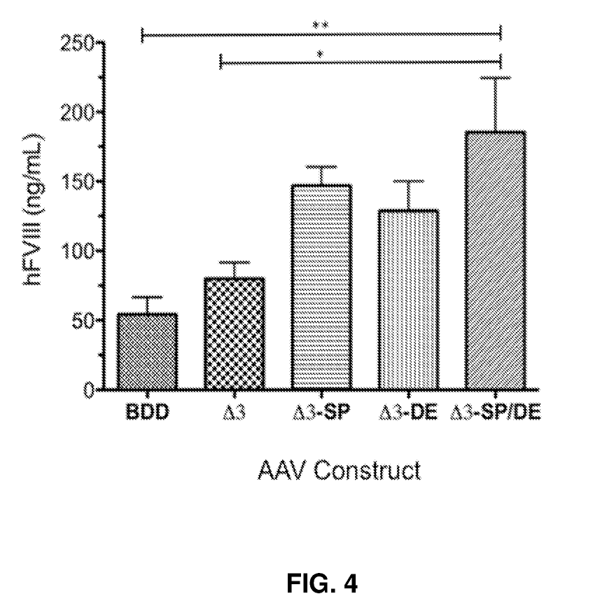

The levels of human FVIII expression at 8 weeks post vector administration in this study (FIG. 4) were: hFVIII-BDD (54.3 ng/ml±12.3), hFVIII-Δ3 (79.8 ng/ml±11.7), hFVIII-Δ3-SP (146.85 ng/ml±13.6), hFVIII-Δ3-DE (128.6 ng/ml±21.4) and hFVIII-A3-SP/DE (185.45 ng/ml±39.1). These data suggest that the hFVIII-A3-SP, hFVIII-A3-DE and hFVIII-A3-SP/DE variants result in higher hFVIII expression after AAV delivery. The hFVIII-A3-SP/DE variant had the highest level of expression. Although not wishing to be bound by any theory, the higher level of expression observed may be due to improved secretion of the hFVIII variants.

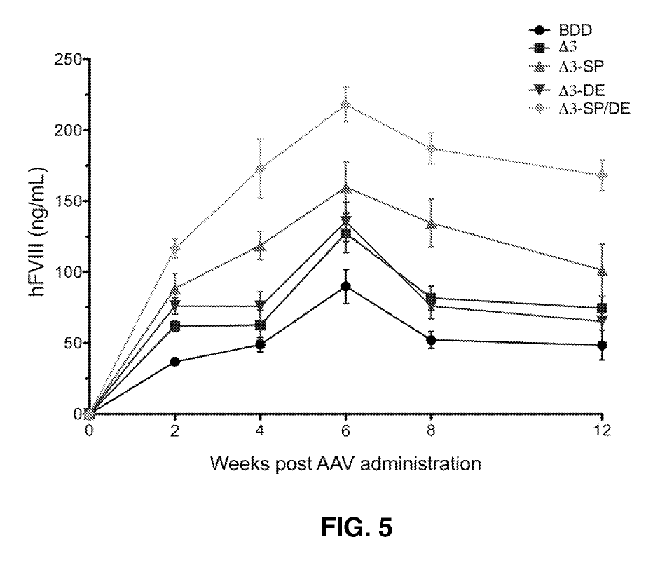

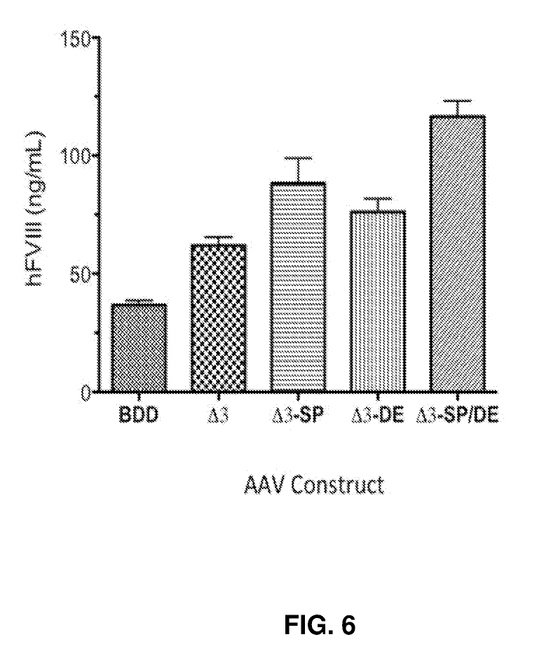

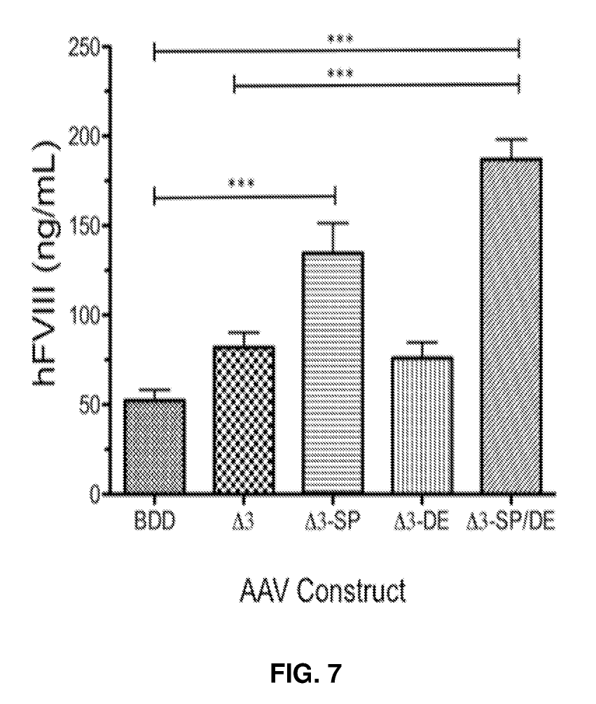

A second study (Study 2) was initiated to confirm the results. Hemophilia A/CD4 knockout (HA/CD4KO) mice were administered 5×10e11vg/mouse and plasma samples were collected at 2, 4 and 8 weeks post vector administration (FIG. 5). At 2 weeks post vector administration, the levels of hFVIII expression were as follows: hFVIII-BDD (36.8 ng/ml±1.9), hFVIII-Δ3 (61.9 ng/ml±3.6), hFVIII-Δ3-SP (88.3 ng/ml±10.6), hFVIII-Δ3-DE (76.1 ng/ml±5.7) and hFVIII-Δ3-SP/DE (116.5 ng/ml±6.7) (FIG. 6). The levels of hFVIII expression at the 8 week time point were as follows: hFVIII-BDD (52.2 ng/ml±6.0), hFVIII-Δ3 (81.8 ng/ml±8.4), hFVIII-Δ3-SP (134.4 ng/ml±16.9), hFVIII-Δ3-DE (76.0 ng/ml±8.8) and hFVIII-Δ3-SP/DE (187.0 ng/ml±11.1) (FIG. 7).

### Example 2

### Expression Studies of the New Variant Proteins.

Expression level studies are shown in Table 1, at 8 weeks (Study 2) and 8 weeks (Study 1). The data show that the SP/DE variant is about 3-4 fold higher than the BDD and 2-fold higher than PACE-furin delta3 variant alone. To summarize, the SP/DE variant is expressed consistently higher than the BDD or Delta 3 (A3) in these studies.

TABLE 1
Comparison of hFVIII Expression Levels
after AAV Delivery of hFVIII Variants

| | hFVIII Expression (ng/ml) | |
|---|---|---|
| Variant | Study 1 (Week 8) | Study 2 (Week 8) |
| hFVIII-BDD | 54.3 ± 12.3 | 52.2 ± 6.0 |
| hFVIII-BDD-Δ3 | 79.8 ± 11.7 | 81.8 ± 8.4 |
| hFVIII-BDD-Δ3-SP | 146.9 ± 13.6 | 134.5 ± 16.9 |
| hFVIII-BDD-Δ3-DE | 128.6 ± 21.4 | 76.0 ± 8.8 |
| hFVIII-BDD-Δ3-SP/DE | 185.5 ± 39.2 | 187.0 ± 11.1 |

### Example 3

### In Vivo Hemostasis Challenge Model.

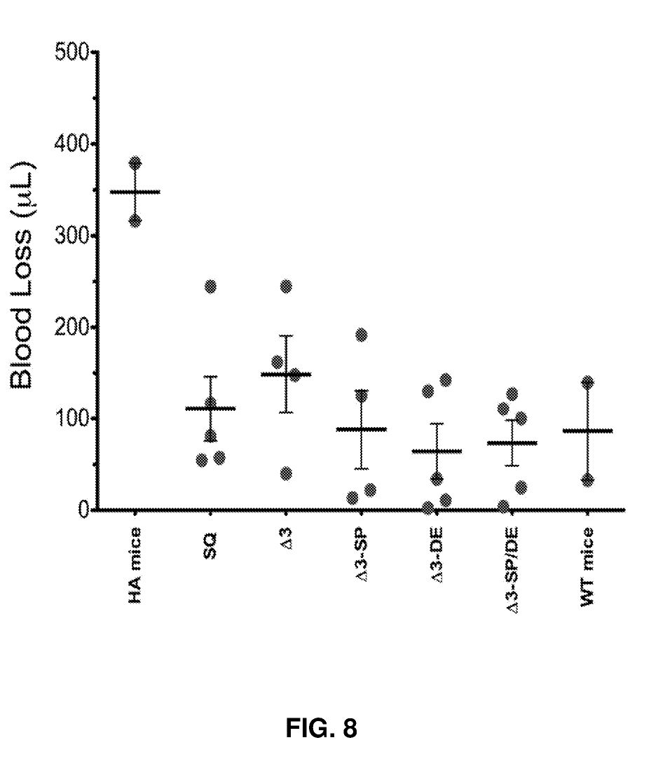

The hemophilia A/CD4 KO mice were challenged in vivo using a complete tail transection model at 6 weeks post AAV vector administration (FIG. 8). The levels of FVIII expression were determined by ELISA (FIG. 7). At the levels of FVIII expression in these mice, the variants (Δ3, Δ3-SP, Δ3-DE, Δ3SP/DE) have blood loss that is similar to hFVIII-BDD and the wild type mice. These results are consistent with previous tail clip assay studies that showed that mice that are expressing between 65-170 ng/ml FVIII from different FVIII transgenes had similar amounts of blood loss (100 μl). At the levels of FVIII expressed in this study, the a3 variants are as effective as hFVIII-BDD at achieving hemostasis. Thus, the variants do not appear different than hFVIII-BDD at the levels of FVIII expression in this study. At lower levels of FVIII expression differences may be observed between the hFVIII-BDD and the a3 variants. Further studies will include the tail clip assay performed after infusion of purified recombinant proteins—a setting in which blood loss can be compared at the same concentration of proteins and the amount of protein can be controlled.

### Factor VIII-BDD (SEQ ID NO:1), SD Residues 1657-58 Bold/Underlined

MQIELSTCFF LCLLRFCFSA TRRYYLGAVE LSWDYMQSDL
GELPVDARFP PRVPKSFPFN TSVVYKKTLF VEFTDHLFNI
AKPRPPWMGL LGPTIQAEVY DTVVITLKNM ASHPVSLHAV
GVSYWKASEG AEYDDQTSQR EKEDDKVFPG GSHTYVWQVL
KENGPMASDP LCLTYSYLSH VDLVKDLNSG LIGALLVCRE
GSLAKEKTQT LHKFILLFAV FDEGKSWHSE TKNSLMQDRD
AASARAWPKM HTVNGYVNRS LPGLIGCHRK SVYWHVIGMG
TTPEVHSIFL EGHTFLVRNH RQASLEISPI TFLTAQTLLM
DLGQFLLFCH ISSHQHDGME AYVKVDSCPE EPQLRMKNNE
EAEDYDDDLT DSEMDVVRFD DDNSPSFIQI RSVAKKHPKT
WVHYIAAEEE DWDYAPLVLA PDDRSYKSQY LNNGPQRIGR
KYKKVRFMAY TDETFKTREA IQHESGILGP LLYGEVGDTL
LIIFKNQASR PYNIYPHGIT DVRPLYSRRL PKGVKHLKDF
PILPGEIFKY KWTVTVEDGP TKSDPRCLIR YYSSFVNMER
DLASGLIGPL LICYKESVDQ RGNQIMSDKR NVILFSVFDE
NRSWYLTENI QRFLPNPAGV QLEDPEFQAS NIMHSINGYV
FDSLQLSVCL HEVAYWYILS IGAQTDFLSV FFSGYTFKHK
MVYEDTLTLF PFSGETVFMS MENPGLWILG CHNSDFRNRG
MTALLKVSSC DKNTGDYYED SYEDISAYLL SKNNAIEPRS
FSQNPPVLKR HQREITRTIL QSDQEEIDYD DTISVEMKKE
DFDIYDEDEN QSPRSFQKKT RHYFIAAVER LWDYGMSSSP
HVLRNRAQSG SVPQFKKVVF QEFTDGSFTQ PLYRGELNEH
LGLLGPYIRA EVEDNIMVTF RNQASRPYSF YSSLISYEED
QRQGAEPRKN FVKPNETKTY FWKVQHHMAP TKDEFDCKAW
AYFSDVDLEK DVHSGLIGPL LVCHTNTLNP AHGRQVTVQE
FALFFTIFDE TKSWYFTENM ERNCRAPCNI QMEDPTFKEN
YRFHAINGYI MDTLPGLVMA QDQRIRWYLL SMGSNENIHS
IHFSGHVFTV RKKEEYKMAL YNLYPGVFET VEMLPSKAGI
WRVECLIGEH LHAGMSTLFL VYSNKCQTPL GMASGHIRDF
QITASGQYGQ WAPKLARLHY SGSINAWSTK EPFSWIKVDL
LAPMIIHGIK TQGARQKFSS LYISQFIIMY SLDGKKWQTY
RGNSTGILMV FFGNVDSSGI KHNIFNPPII ARYIRLHPTH
YSIRSTLRME LMGCDLNSCS MPLGMESKAI SDAQITASSY
FTNMFATWSP SKARLHLQGR SNAWRPQVNN PKEWLQVDFQ
KTMKVTGVTT QGVKSLLTSM YVKEFLISSS QDGHQWTLFF
QNGKVKVFQG NQDSFTPVVN SLDPPLLTRY LRIHPQSWVH
QIALRMEVLG CEAQDLY

### Canine Factor VIII-BDD (SEQ ID NO:2), PACE/Furin Region 1637-1655, HHQR (1637-1648) and PE (1649 and 1650) Bold/Underlined

MQVELYTCCFLCLLPFSLSATRKYYLGAVELSWDYMQSDLLSALHADT
SFSSRVPGSLPLTTSVTYRKTVFVEFTDDLFNIAKPRPPWMGLLGPTI
QAEVYDTVVIVLKNMASHPVSLHAVGVSYWKASEGAEYEDQTSQKEKE
DDNVIPGESHTYVWQVLKENGPMASDPPCLTYSYFSHVDLVKDLNSGL
IGALLVCKEGSLAKERTQTLQEFVLLFAVFDEGKSWHSETNASLTQAE
AQHELHTINGYVNRSLPGLTVCHKRSVYWHVIGMGTTPEVHSIFLEGH
TFLVGNHRQASLEISPITFLTAQTFLMDLGQFLLFCHIPSHQHDGMEA
YVKVDSCPEEPQLRMKNNEDKDYDDGLYGSDMDVVSFDDDSSSPFIQI
RSVAKKHPKTWVHYIAAEEEDWDYAPSGPTPNDRSHKNLYLNNGPQRI
GKKYKKVRFVAYTDETFKTREAIQYESGILGPLLYGEVGDTLLIIFKK
QASRPYNIYPHGINYVTPLHTGRLPKGVKHLKDMPILPGEIFKYKWTV
TVEDGPTKSDPRCLTRYYSSFINLERDLASGLIGPLLICYKESVDQRG
NQMMSDKRNVILFSVLDENRSWYLTEDMQRFLPNADVVQPHDPEFQLS
NIMHSINGYVFDNLQLSVCLHEVAYWYILSVGAQTDFLSVFFSGYTFK
HKMVYEDTLTLFPFSGETVFMSMENPGLWVLGCHNSDFRNRGMTALLK
VSSCNRNIDDYYEDTYEDIPTPLLNENNVIKPRSFSQNPPVSKHHQRE
ITVTTLQPEEDKFEYDDTFSIEMKREDFDIYGDYEDQGLRSFQKKTRH
YFIAAVERLWDYGMSRSPHILRNRAQSGDVQQFKKVVFQEFTDGSFTQ
PLYRGELNEHLGLLGPYIRAEVEDNIVVTFKNQASRPYSFYSSLISYD
EDEGQGAEPRRKFVNPNETKIYFWKVQHHMAPTKDEFDCKAWAYFSDV
DLEKDVHSGLIGPLLICRSNTLNPAHGRQVTVQEFALVFTIFDETKSW
YFTENLERNCRAPCNVQKEDPTLKENFRFHAINGYVKDTLPGLVMAQD
QKVRWYLLSMGSNENIHSIHFSGHVFTVRKKEEYKMAVYNLYPGVFET
VEMLPSQVGIWRIECLIGEHLQAGMSTLFLVYSKKCQTPLGMASGHIR
DFQITASGQYGQWAPKLARLHYSGSINAWSTKDPFSWIKVDLLAPMII
HGIMTQGARQKFSSLYVSQFIIMYSLDGNKWHSYRGNSTGTLMVFFGN
VDSSGIKHNIFNPPIIAQYIRLHPTHYSIRSTLRMELLGCDFNSCSMP
LGMESKAISDAQITASSYLSSMLATWSPSQARLHLQGRTNAWRPQANN
PKEWLQVDFRKTMKVTGITTQGVKSLLISMYVKEFLISSSQDGHNWTL
FLQNDKVKVFQGNRDSSTPVRNALEPPLVARYVRLHPQSWAHHIALRL
EVLGCDTQQPA

### Representative Non-Limiting Variants of Canine FVIII:

| - = deletion of residue | |
|---|---|
| HHQREITVTTLQPEEDKFEYDD | wild type sequence |
| ---REITVTTLQ--EDKFEYDD | |
| ---REITV------EDKFEYDD | |
| ---REITVTTLQ------EYDD | |

### Porcine Factor VIII-BDD (SEQ ID NO:3), PACE/Furin Region 1427-1445, RHQR (1427-1430) and PE (1439 and 1440) Bold/Underlined

MQLELSTCVFLCLLPLGFSAIRRYYLGAVELSWDYRQSELLRELHVDT
RFPATAPGALPLGPSVLYKKTVFVEFTDQLFSVARPRPPWMGLLGPTI
QAEVYDTVVVTLKNMASHPVSLHAVGVSFWKSSEGAEYEDHTSQREKE
DDKVLPGKSQTYVWQVLKENGPTASDPPCLTYSYLSHVDLVKDLNSGL
IGALLVCREGSLTRERTQNLHEFVLLFAVFDEGKSWHSARNDSWTRAM
DPAPARAQPAMHTVNGYVNRSLPGLIGCHKKSVYWHVIGMGTSPEVHS
IFLEGHTFLVRHHRQASLEISPLTFLTAQTFLMDLGQFLLFCHISSHH
HGGMEAHVRVESCAEEPQLRRKADEEEDYDDNLYDSDMDVVRLDGDDV
SPFIQIRSVAKKHPKTWVHYISAEEEDWDYAPAVPSPSDRSYKSLYLN
SGPQRIGRKYKKARFVAYTDVTFKTRKAIPYESGILGPLLYGEVGDTL
LIIFKNKASRPYNIYPHGITDVSALHPGRLLKGWKHLKDMPILPGETF
KYKWTVTVEDGPTKSDPRCLTRYYSSSINLEKDLASGLIGPLLICYKE
SVDQRGNQMMSDKRNVILFSVFDENQSWYLAENIQRFLPNPDGLQPQD
PEFQASNIMHSINGYVFDSLQLSVCLHEVAYWYILSVGAQTDFLSVFF
SGYTFKHKMVYEDTLTLFPFSGETVFMSMENPGLWVLGCHNSDLRNRG
MTALLKVYSCDRDIGDYYDNTYEDIPGFLLSGKNVIEPRSFAPKPPVL
RRHQRDISLPTFQPEEDKMDYDDIFSTETKGEDFDIYGEDENQDPRSF
QKRTRHYFIAAVEQLWDYGMSESPRALRNRAQNGEVPRFKKVVFREFA
DGSFTQPSYRGELNKHLGLLGPYIRAEVEDNIMVTFKNQASRPYSFYS
SLISYPDDQEQGAEPRHNFVQPNETRTYFWKVQHHMAPTEDEFDCKAW
AYFSDVDLEKDVHSGLIGPLLICRANTLNAAHGRQVIVQEFALFFTIF
DETKSWYFTENVERNCRAPCHLQMEDPTLKENYRFHAINGYVMDTLPG
LVMAQNQRIRWYLLSMGSNENIHSIHFSGHVFSVRKKEEYKMAVYNLY
PGVFETVEMLPSKVGIWRIECLIGEHLQAGMSTTFLVYSKECQAPLGM
ASGRIRDFQITASGQYGQWAPKLARLHYSGSTHAWSTKDPHSWIKVDL
LAPMIIHGIMTQGARQKFSSLYISQFIIMYSLDGRNWQSYRGNSTGTL
MVFFGNVDASGIKHNIFNPPIVARYIRLHPTHYSIRSTLRMELMGCDL
NSCSMPLGMQNKAISDSQITASSHLSNIFATWSPSQARLHLQGRTNAW
RPRVSSAEEWLQVDLQKTVKVTGITTQGVKSLLSSMYVKEFLVSSSQD
GRRWTLFLQDGHTKVFQGNQDSSTPVVNALDPPLFTRYLRIHPTSWAQ
HIALRLEVLGCEAQDLY

### Representative Non-Limiting Variants of Porcine FVIII:

- = deletion of residue

| | |
|---|---|
| RHQRDISLPTFQPEEDKMDYDD | wild type sequence |
| ---RDISLPTFQ--EDKMDYDD | |
| ---RDISL------EDKMDYDD | |
| ---RDISLPTFQ------DYDD | |

### Example 4

### Protein Purification of a3 Variants.

Stable BHK clones were generated that express the hFVIII-Δ3-SP, hFVIII-Δ3-DE and hFVIII-Δ3-SP/DE variants. Previously, stable BHK clones were established for the hFVIII-BDD and hFVIII-Δ3 constructs. After transfection and selection, 50 clones of each variant were isolated and screened to identify the three best clones of each construct. The screening was performed by assaying the media for FVIII using a clotting assay (activated partial thromboplastin time, aPTT) and an ELISA to determine the antigen levels. The best clone for each variant is shown in Table 2. hFVIIIΔ3-DE and hFVIII-Δ3-SP/DE have levels of expression and activity that is similar to hFVIII-Δ3. These data suggest that a3 variant activity may be similar to hFVIII-Δ3. Recombinant protein was purified to characterize the procoagulant activity and function in vitro and in vivo.

TABLE 2
Characterization of hFVIII Variant Stable BHK Clones

| | | Human FVIII Variant | | | |
|---|---|---|---|---|---|
| Assay | Δ3-SP | Δ3-DE | Δ3-SP/DE | Δ3 | BDD |
| ELISA (ng/mL) | 680 | 1286 | 921 | 1310 | 767.8 |
| APTT (sec) | 42.4 | 34.6 | 32.6 | 36.9 | 46 |

Two a3 variant proteins have been purified: (1) SP/DE and (2) Δ3-SP/DE. Based on the studies of AAV delivery of these variants, the best variant was selected based on the improved factor VIII expression, Δ3-SP/DE, to purify and characterize. The Δ3-SP/DE variant has the A3 furin deletion as well as the SP/DE amino acid change in the a3 region (shown in Table 3). The SP/DE variant does not include the A3 deletion but only the SP/DE modification. This SP/DE variant was also purified to understand the role of this site independent of the furin modification. These variants have been introduced into the wild type B-domain deleted human factor VIII sequence (hFVIII).

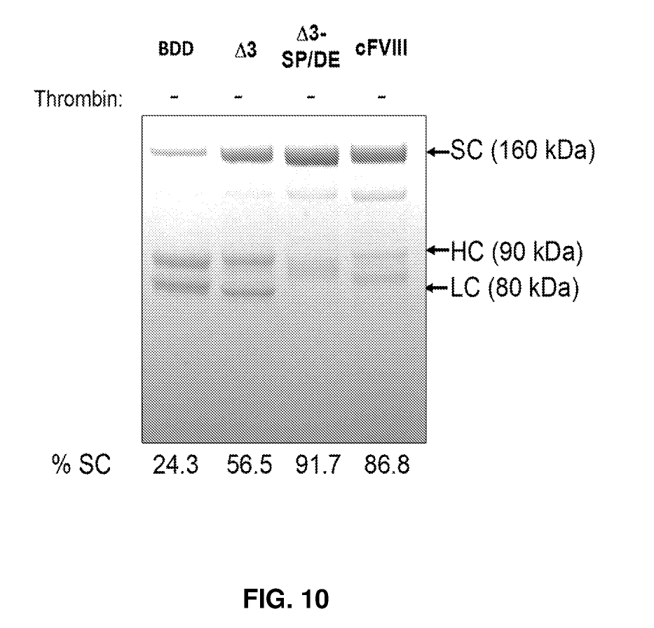

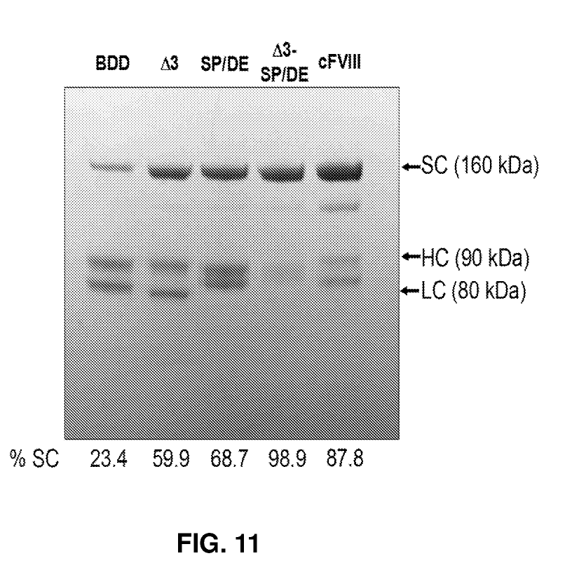

To determine the protein structure of the purified proteins, variant FVIII proteins were analyzed on an SDS-PAGE gel in two separate studies (FIGS. 10 and 11). Wild type B-domain deleted human FVIII (hFVIII-BDD) is secreted primarily as a heterodimer composed of the heavy chain (HC) and the light chain (LC) with some protein that remains in the uncleaved hFVIII single polypeptide chain (SC). However, these gels show that there is an increase in the amount of the protein in the single chain polypeptide form when the furin deletion variant (Δ3) or the SP/DE variant is introduced into the wild type human FVIII protein sequence (hFVIII-BDD).

More specifically, FIG. 10 shows that the A3 and the Δ3-SP/DE variants have an increase in the single chain polypeptide form (57% and 92%, respectively) compared to hFVIII-BDD (24%). Analysis of the SP/DE variant alongside the A3 and Δ3-SP/DE variants on an SDS-PAGE gel (FIG. 11) demonstrated that the SP/DE variant (69%) was similar to the A3 variant alone (60%) while the Δ3-SP/DE variant was 98% in the single chain form. Upon activation with thrombin, all variants generate the expected protein species which demonstrates that these variants are all cleaved by thrombin to form the active protein. Together these data indicate that the combined A3 and SP/DE variants (Δ3-SP/DE) results in a decrease in cleavage of the protein and consequently an increase in the single chain form of the protein.

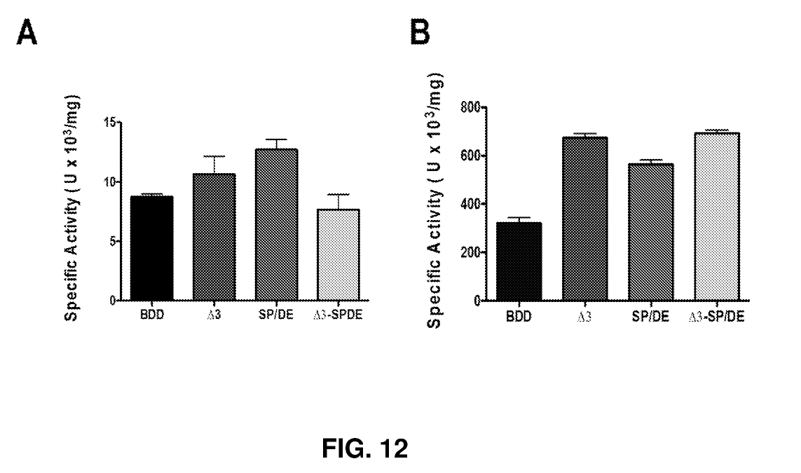

The activity of the FVIII variants was determined using an activated partial thromboplastin time (aPTT) assay (FIG. 12). In a one-stage aPTT assay the variants have similar activity to hFVIII-BDD. However, in a two-stage aPTT assay in which the protein is activated with thrombin before the clotting assay is initiated, all variant proteins (Δ3, SP/DE and Δ3-SP/DE) have 2-fold higher activity compared to hFVIII-BDD. The Δ3-SP/DE variant had higher biological activity compared to hFVIII-BDD but the activity was similar to A3 or SP/DE alone.

Ongoing studies will seek to understand the reason for this increased activity. In addition, these proteins will be infused into the hemophilia A mice and a tail clip assay performed to determine if these proteins provide enhanced function in vivo. Although not being bound, the prediction is that Δ3-SP/DE and SP/DE may be similar to A3 in achieving hemostasis which are all better than hFVIII-BDD.

### Example 5

### Delivery of AAV Vectors Expressing the FVIII a3 Variants

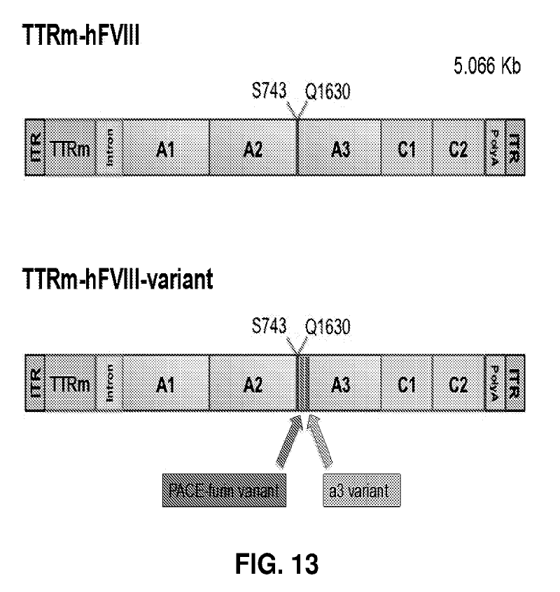

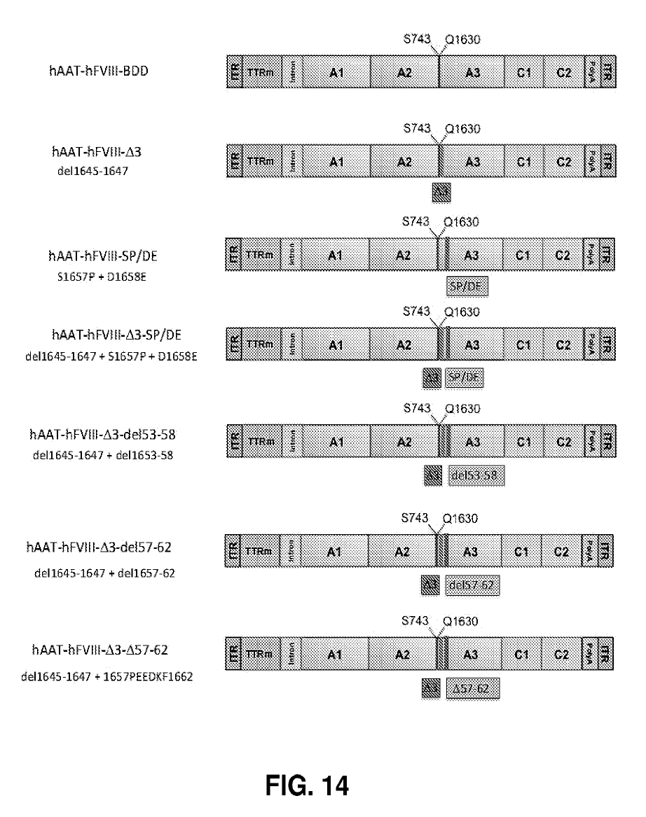

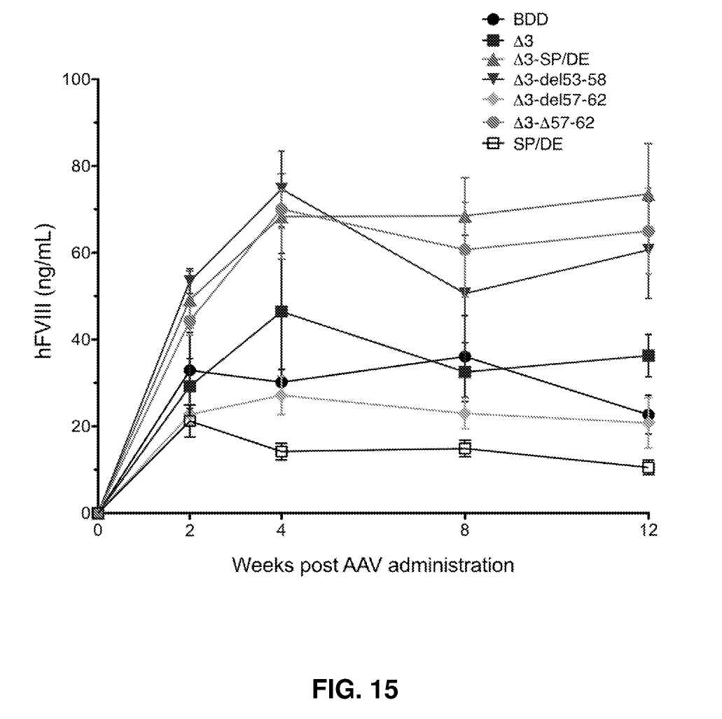

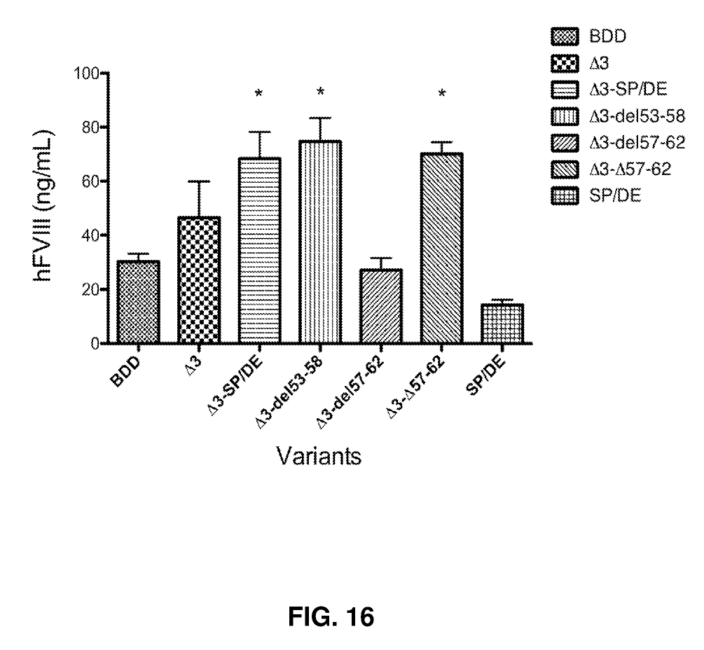

Additional hFVIII variants of the a3 site have been introduced into an AAV8-hFVIII transgene construct (Table 3, last 4 FVIII variants). In these studies, an optimized expression cassette was used that utilizes a different promoter, a modified transthyretin promoter (TTRm)(FIG. 13). The hFVIII variants (Δ3, SP/DE, Δ3-SP/DE, Δ3-del53-58, Δ3-del57-62, and Δ3-Δ57-62) were introduced into the wild type B-domain deleted hFVIII sequence (hFVIII-BDD)(FIG. 14). Expression from this optimized expression cassette is 4-5-fold higher than the expression cassette used in the initial studies (Example 1). The advantage of using this optimized expression cassette is that a lower AAV vector dose can be used to achieve similar levels of hFVIII expression. In this study, AAV8-hFVIII (1×10e11vg/mouse) was administered to hemophilia A/CD4 KO mice (n=4/group). Plasma samples were collected at 2, 4, 8 and 12 weeks post-vector administration and an ELISA was used to determine the hFVIII antigen levels (FIG. 15). Levels of expression at 4 weeks post vector administration (FIG. 16) were: hFVIII-BDD (30.2 ng/ml±3.0), Δ3 (46.5 ng/ml±13.4), Δ3-SP/DE (68.35 ng/ml±9.9), Δ3-del53-58 (74.7 ng/ml±8.7), Δ3-del57-62 (27.1 ng/ml±4.5), Δ3-A57-62 (70.1 ng/ml±4.4) and SP/DE (14.2 ng/ml±1.9). These data show that the Δ3-SP/DE, Δ3-del53-58 and Δ3-Δ57-62 express higher hFVIII levels than FVIII-BDD or FVIII-A3 alone.

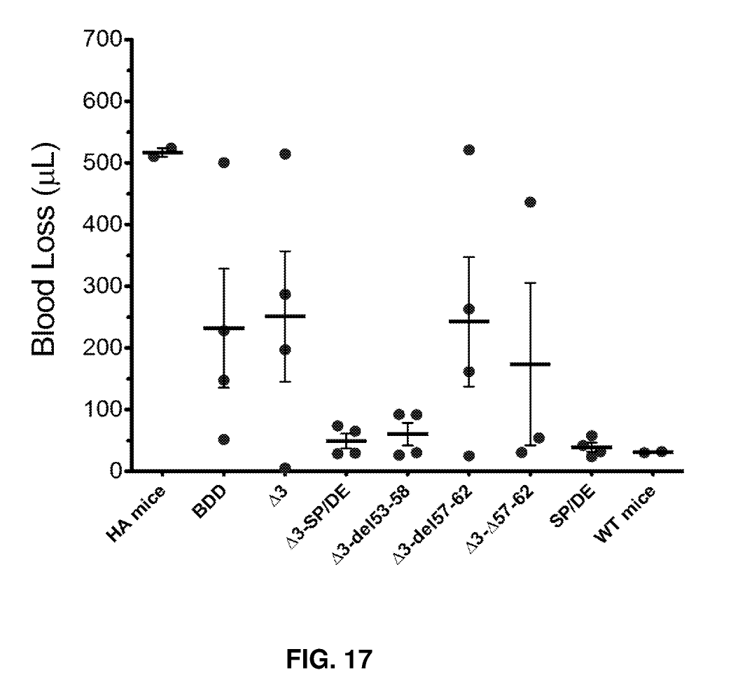

At 6 weeks post vector administration, a tail clip assay was performed to evaluate the in vivo function of these variants (FIG. 17). In this assay, a tail transection is performed at 3 mm diameter in the mouse's tail and blood is collected in warm saline for 10 minutes. The sample is centrifuged and the red blood cells are lysed to measure the hemoglobin content by absorbance at 575 nm compared to a standard curve to determine blood loss. In this assay the amount of blood loss is affected by the levels of FVIII expression but also the function of the protein. Untreated hemophilia A mice have significant blood loss (500 μl) whereas wild type mice have little blood loss (3 μl).

The hemophilia A mice treated with the AAV8-hFVIII constructs (FIG. 17) have different amounts of blood loss. The hFVIII-BDD, Δ3, Δ3-del57-62 FVIII variants and Δ3-Δ57-62 had partial correction of the bleeding phenotype. It is common to observe high variability within a treatment group when levels of FVIII in the circulation are not sufficient to fully correct the bleeding phenotype. In contrast, mice administered Δ3-SP/DE, Δ3-del53-58 and SP/DE FVIII variants had minimal blood loss that was similar to the wild type mice. Interestingly, SP/DE did not result in high levels of FVIII expression (FIGS. 15 and 16) but was very effective at minimizing blood loss after the tail clip assay. Thus, these data indicate that at this AAV vector dose, the Δ3-SP/DE, Δ3-del53-58 and SP/DE FVIII variants were superior to the other variants at restoring hemostasis.

### Example 6

Table 3 summarizes numerous representative FVIII variants disclosed herein. Data on AAV delivery of the FVIII variants as well as biochemical characterization of the purified protein are disclosed herein.

TABLE 3
Summary of variants in human factor VIII.

| | | Furin recognition site and acidic region 3 (a3) site (1645-1662) | | | | | | | | | | | | | | | | | |
|---|---|---|---|---|---|---|---|---|---|---|---|---|---|---|---|---|---|---|---|
| hFVIII Variant | | 45 | 46 | 47 | 48 | 49 | 50 | 51 | 52 | 53 | 54 | 55 | 56 | 57 | 58 | 59 | 60 | 61 | 62 |
| hfVIII | BDD | R | H | Q | R | E | I | T | R | T | T | L | Q | S | D | Q | E | E | I |
| cfVIII | cfVIII | H | H | Q | R | E | I | T | V | T | T | L | Q | P | E | E | D | K | F |
| hFVIII-Δ3 | Δ3 | — | — | — | R | E | I | T | R | T | T | L | Q | S | D | Q | E | E | I |
| hFVIII-S1657P/D1658E | SP/DE | R | H | Q | R | E | I | T | R | T | T | L | Q | P | E | Q | E | E | I |
| hFVIII-Δ3-S1657P | Δ3-SP | — | — | — | R | E | I | T | R | T | T | L | Q | P | D | Q | E | E | I |
| hFVIII-Δ3-D1658E | Δ3-DE | — | — | — | R | E | I | T | R | T | T | L | Q | S | E | Q | E | E | I |
| hFVIII-Δ3-S1657P/D1658E | Δ3-SP/DE | — | — | — | R | E | I | T | R | T | T | L | Q | P | E | Q | E | E | I |
| hFVIII-Δ3-del1657-58 | Δ3-del57-58 | — | — | — | R | E | I | T | R | T | T | L | Q | — | — | Q | E | E | I |
| hFVIII-Δ3-del1653-58 | Δ3-del53-58 | — | — | — | R | E | I | T | R | — | — | — | — | — | — | Q | E | E | I |
| hFVIII-Δ3-del1657-62 | Δ3-del57-62 | — | — | — | R | E | I | T | R | T | T | L | Q | — | — | — | — | — | — |
| hFVIII-Δ3-1657PEEDKF1662 | Δ3-Δ57-62 | — | — | — | R | E | I | T | R | T | T | L | Q | P | E | E | D | K | F |

### Example 7

### Further Definitions/Abbreviations Used

hFVIII-BDD (SQ): human FVIII with B domain deletion
hFVIII-Δ3 or Δ3: human FVIII with B domain deletion; deletion of amino acids at positions 1645, 1646 and 1647 of human FVIII
hFVIII-Δ3-SP or Δ3-SP: human FVIII with B domain deletion; deletion of amino acids at positions 1645, 1646 and 1647 of FVIII and S1657P substitution (serine->proline) of FVIII.
hFVIII-Δ3-DE or Δ3-DE: human FVIII with B domain deletion; deletion of amino acids at positions 1645, 1646 and 1647 of FVIII and D1658E (aspartic acid->glutamic acid) substitution of FVIII.
hFVIII-Δ3-SP/DE or Δ3-SP/DE: human FVIII with B domain deletion; deletion of amino acids at positions 1645, 1646 and 1647 of FVIII and S1657P (serine->proline) and D1658E (aspartic acid->glutamic acid) substitution of FVIII.
As used herein, certain amino acid positions of FVIII are referenced. For the sake of brevity, in certain instances reference to a particular species of FVIII is omitted. In such instances, positions 1657 or 1658 of FVIII protein typically refer to human FVIII. In such instances, positions 1645 through 1662 of FVIII protein typically refer to human FVIII.
In such instances, positions 1649 or 1650 of FVIII protein typically refer to canine FVIII. In such instances, positions 1637 through 1655 of FVIII protein typically refer to canine FVIII. In such instances, positions 1439 or 1440 of FVIII protein typically refer to porcine FVIII. In such instances, positions 1427 through 1445 of FVIII protein typically refer to porcine FVIII.

While certain of the embodiments of the invention have been described and specifically exemplified above, it is not intended that the invention be limited to such embodiments. Various modifications may be made thereto without departing from the scope and spirit of the invention, as set forth in the following claims.

1. (canceled)

2. A Factor VIII (FVIII) variant, wherein the FVIII variant comprises a B domain deletion and has one or more amino acids at positions 1645 through 1662 of FVIII protein substituted, modified or deleted, compared to wild type FVIII comprising a B domain deletion.

3. The Factor VIII (FVIII) variant of claim 2, wherein the FVIII variant has 1 or 2 amino acids at positions 1657 or 1658 of FVIII protein substituted, modified or deleted compared to wild type FVIII comprising a B domain deletion.

4.-5. (canceled)

6. The Factor VIII (FVIII) variant of claim 2, wherein the FVIII variant comprises a B domain deletion and has 1 to 6 amino acids at positions 1653 to 1658 of FVIII protein substituted, modified or deleted, compared to wild type FVIII comprising a B domain deletion.

7. The Factor VIII (FVIII) variant of claim 2, wherein the FVIII variant comprises a B domain deletion and has 1 to 6 amino acids at positions 1657 through 1662 of FVIII protein substituted, modified or deleted, compared to wild type FVIII comprising a B domain deletion.

8. The Factor VIII (FVIII) variant of claim 2, wherein the FVIII variant has 1 to 10 amino acids at positions 1653 to 1662 of FVIII protein substituted, modified or deleted compared to wild type FVIII comprising a B domain deletion.

9. The Factor VIII (FVIII) variant of claim 2, wherein the FVIII protein with 1 to 6 amino acids substituted, modified or deleted is a human, canine or porcine FVIII.

10. The Factor VIII (FVIII) variant of claim 2, wherein the FVIII protein with 1 to 6 amino acids substituted, modified or deleted is a wild type human, canine or porcine FVIII.

11.-18. (canceled)

19. The FVIII variant of claim 2, wherein said variant exhibits greater activity or expression levels when compared to activity or expression levels of wild type FVIII or wild type FVIII comprising a B domain deletion.

20. The FVIII variant of claim 2, wherein said variant is more efficiently secreted by a cell in which it is expressed compared to secretion of a wild type FVIII or wild-type FVIII comprising a B domain deletion.

21. The FVIII variant of claim 2, wherein said variant is secreted by a cell in which it is expressed at least 1.5-5-fold higher than secretion of a wild type FVIII or wild-type FVIII comprising a B domain deletion.

22. The FVIII variant of claim 2, wherein said variant is secreted by a cell in which it is expressed at least 1.5-3-fold higher than secretion of a wild type FVIII or wild-type FVIII comprising a B domain deletion wherein 1, 2, 3 or all 4 of the amino acids comprising the PACE/furin cleavage site set forth as HHQR or RHQR from positions 1645-1648 in the wild type FVIII or wild-type FVIII comprising a B domain deletion is/are substituted, modified or deleted.

23.-25. (canceled)

26. The FVIII variant of claim 2, wherein said variant has both amino acids at positions 1657 and 1658 of human FVIII protein substituted, modified or deleted.

27. The FVIII variant of claim 2, wherein said variant has a conservative amino acid substitution at amino acids at positions 1657 and/or 1658 of human FVIII protein.

28. The FVIII variant of claim 2, wherein said variant has a proline at position 1657, 1649 and/or 1439.

29. (canceled)

30. The FVIII variant of claim 2, wherein said variant has a glutamic acid at position 1658, 1650 and/or 1440.

31. (canceled)

32. The FVIII variant of claim 2, wherein said variant has a proline at position 1657 and an aspartic acid at position 1658, 1650 and/or 1440.

33.-36. (canceled)

37. The FVIII variant of claim 2, wherein said variant has a threonine deleted at position 1653; and/or wherein said variant has a threonine deleted at position 1654; and/or wherein said variant has a leucine deleted at position 1655; and/or, wherein said variant has a glutamine deleted at position 1656.

38. (canceled)

39. The FVIII variant of claim 2, wherein said variant has a glutamic acid deleted at position 1659; and/or wherein said variant has an aspartic acid deleted at position 1660; and/or wherein said variant has a lysine deleted at position 1661;
and/or, wherein said variant has a phenylalanine deleted at position 1662.

40. The FVIII variant of claim 2, wherein 1, 2, 3 or all 4 of the amino acids comprising the PACE/furin cleavage site is/are substituted, modified or deleted.

41.-44. (canceled)

45. The FVIII variant of claim 2, wherein the variant has the amino acid substitutions and/or deletions shown in Table 3 denoted as hFVIII-Δ3; hFVIII-S1657P/D1658E (SP/DE); hFVIII-Δ3-S1657P (Δ3-SP); hFVIII-Δ3-D1658E (Δ3-DE); hFVIII-Δ3-S1657P/D1658E (Δ3-SP/DE); hFVIII-Δ3-del1657-58 (Δ3-del57-58); hFVIII-Δ3-del1653-58 (Δ3-del53-58); hFVIII-Δ3-del1657-62 (Δ3-del57-62); or hFVIII-Δ3-del1657PEEDKF1662 (Δ3-Δ57-62).

46.-47. (canceled)

48. The FVIII variant of claim 2, wherein said variant is at least 75% identical to wild type human FVIII or wild type human FVIII comprising a B domain deletion.

49. The FVIII variant of claim 2, wherein said variant is a mammalian FVIII, comprising a B domain deletion is a mammalian FVIII.

50. (canceled)

51. A nucleic acid encoding the Factor VIII (FVIII) variant of claim 2.

52.-71. (canceled)

72. A host cell expressing the FVIII variant of claim 2.

73.-78. (canceled)

79. A pharmaceutical composition comprising the FVIII of claim 2 in a biologically compatible carrier or excipient.

80.-101. (canceled)
